(19) United States
(12) Patent Application Publication
(10) Pub. No.: US 2025/0263792 A1
(43) Pub. Date: Aug. 21, 2025

(54) METHODS FOR THE IDENTIFICATION AND TREATMENT OF SEVERE FORMS OF COVID-19

(71) Applicant: Genuity Science, Inc., Boston, MA (US)

(72) Inventors: Seiamak BAHRAM, Strasbourg (FR); Thomas W. CHITTENDEN, Medford, MA (US); Raphael CARAPITO, Strasbourg (FR)

(73) Assignee: Genuity Science, Inc., Boston, MA (US)

(21) Appl. No.: 18/560,221

(22) PCT Filed: May 9, 2022
(86) PCT No.: PCT/US2022/028331

§ 371 Date: Nov. 10, 2023

Related U.S. Application Data
(60) Provisional application No. 63/186,560, filed on May 10, 2021.

(51) Int. Cl. C12Q 1/6883; A61P 31/14; C12N 15/113; G16B 40/00; G16H 50/20

(52) U.S. Cl. CPC C12Q 1/6883; A61P 31/14; C12N 15/113; G16B 40/00; G16H 50/20; C12N 2310/14; C12Q 2600/106; C12Q 2600/118; C12Q 2600/156; C12Q 2600/158

(57) ABSTRACT

Provided herein are method for treating or preventing severe coronavirus disease 2019 (COVID-19) in a subject, comprising administering to the subject a composition comprising a modulating agent that decreases or increases the expression or gene product activity of one or more driver genes.

### CROSS-REFERENCE TO RELATED APPLICATIONS

This application is a national phase application that claims priority from PCT application PCT/US2022/028331 filed May 9, 2022, which claims the benefit of U.S. Provisional Application No. 63/186,560, filed May 10, 2021. These applications are hereby incorporated by reference in their entirety.

### STATEMENT REGARDING SEQUENCE LISTING

The Sequence Listing associated with this application is provided in text format and is hereby incorporated by reference into the specification. The name of the text file containing the sequence listing is 2095-P366US.PNP_SequenceListing_ST25.txt. The text file is 1,079 bytes; was created on Nov. 8, 2023; and is being submitted electronically via patent Center with the filing of the specification.

### BACKGROUND

Unlike many viral infections and most respiratory virus infections, COVID-19 displays an extraordinary complex and diversified spectrum of clinical manifestations, hence the naming of “syndemic” within, or in lieu of, a pandemic (Horton, 2020). Indeed, upon infection by SARS-CoV-2, age, sex, and phenotypically-matched individuals can evolve schematically within four distinct groups, i.e., those (1) being asymptomatic, (2) displaying influenza-like-illnesses, (3) affected by respiratory dysfunction eventually needing external oxygen supply, and (4) afflicted with an acute respiratory distress syndrome (ARDS) requiring mechanical ventilation in an intensive care unit (ICU). Despite the fact that the last group represents only a small fraction of COVID-19 patients, it encompasses the most severe form of disease with an average case-fatality rate of around 25% (Quah et al., 2020).

Several studies have used multiple omics technologies to uncover key molecular processes associated with disease severity. Systemic inflammation with high levels of acute phase proteins (CRP, SAA, calprotectin) (Silvin et al., 2020) and inflammatory cytokines, particularly interleukin (IL)-6 and IL-1β (Chen et al., 2020a; Giamarellos-Bourboulis et al., 2020; Lucas et al., 2020) have been shown to be a hallmark of disease severity. In contrast, following an initial burst shortly after infection, the type I interferon response was shown to be impaired at the RNA (Hadjadj et al., 2020), plasma (Trouillet-Assant et al., 2020) and genetic level (Zhang et al., 2020). Severity was also shown to be correlated with profound immune dysregulations including modifications in the myeloid compartment with increases in neutrophils (Meizlish et al., 2021; Schulte-Schrepping et al., 2020), decreases in non-classical monocytes (Silvin et al., 2020) and dysregulation of macrophages (Giamarellos-Bourboulis et al., 2020; Shen et al., 2020). The lymphoid compartment was also shown to be modified with both a B-cell response activation (De Biasi et al., 2020a) and an impaired T-cell response characterized by a skewing towards a Th17 phenotype (De Biasi et al., 2020b; Odak et al., 2020). Finally, coagulation defects have been identified in critically ill patients that are prone to thrombotic complications (Klok et al., 2020). Nevertheless, not a single study has applied the full spectrum of omics technology to a highly curated COVID-19 patients and controls dataset where a number of key confounding factors that affect severity and death such as older age and comorbidities have been discarded at the onset.

Despite intense investigation, the fundamental question as to why the course of the disease differs so greatly is largely unanswered (The Severe Covid-19 GWAS, 2020; Zhang et al., 2020); i.e., the exact pathophysiological mechanisms governing disease severity within a demographically and clinically homogeneous group of patients is still unclear. To better understand this, there is a need for high-resolution molecular analyses applied on well-defined cohorts of patients and controls.

### SUMMARY

The pathogenesis of severe forms of COVID19, especially in young patients, remains a salient unanswered question. Without being bound by theory, it is hypothesized that SARS-CoV-2 induces characteristic molecular changes in critical patients that can be used to differentiate them from non-critical patients. The present invention is based, at least in part, on the discovery that certain driver genes may also be responsible for the development of critical illness, and such genes may represent therapeutic targets. As disclosed herein, ensemble artificial intelligence/machine learning-based multi-omics studies were performed on young (<50 years of age) COVID-19 patients without major comorbidities admitted to the ICU and under mechanical ventilation (“critical patients”) versus matched COVID-19 patients needing only hospitalization in a non-critical care ward (25 “non-critical patients”); and an age- and sex-matched control group of healthy non-COVID-19 individuals. The multi-omics approaches disclosed herein included Whole Genome Sequencing (WGS), whole blood RNA-sequencing (RNA-seq), quantitative plasma and Peripheral Blood Mononuclear Cells (PBMC) proteomics, multiplex plasma cytokine profiling and high throughput immune cells phenotyping in conjunction with viral parameters i.e., anti-SARS-Cov-2 neutralizing antibodies and multi-target antiviral serology. Provided herein are are unique gene signatures that differentiate critical from non-critical patients as identified by an ensemble of machine learning, deep learning and quantum annealing methods. Within such gene signatures, structural causal modeling can identify driver genes that may promote ARDS etiology. For example, and without limitation, the up-regulated metalloprotease ADAM9 is identified as a key driver. Inhibition of ADAM9 ex vivo interfered with SARS-Cov-2 uptake and replication in human epithelial cells. In brief, an advanced integrated machine learning and probabilistic programming strategy was applied to identify causal molecular drivers of severe forms of COVID-19 in a small, tightly controlled cohort of patients, the importance of which were then experimentally validated.

In some aspects of the disclosed invention, provided herein are methods for treating or preventing severe coronavirus disease 2019 (COVID-19) in a subject, comprising administering to the subject a composition comprising modulating agents of ADAM9, MCEMP1, MS4A4A, RAB10, GCLM, EPHX2, RORA, CFAP97, ARL4C, or ACSS1. Modulating agents may decrease or increase the activity or level of the corresponding gene products (e.g., transcript and/or protein).

In some aspects of the invention, provided herein are methods of treating and/or preventing severe COVID-19 in a subject. In further aspects, provided herein are methods for predicting the likelihood of a subject infected with SARS-CoV-2 to progressing to severe COVID-19. In some embodiments, such methods include (a) sequencing at least part of the subject's genome in a sample from said subject, wherein the at least part of said genome comprises an ADAM9, MCEMP1, MS4A4A, RAB10, GCLM, EPHX2, RORA, CFAP97, ARL4C, or ACSS1 gene; (b) identifying from the sequencing of said sample at least one at least one single-nucleotide polymorphism (SNP in one or more of genes: ADAM9, MCEMP1, MS4A4A, RAB10, GCLM, EPHX2, RORA, CFAP97, ARL4C, or ACSS1; and (c) administering a corresponding modulating agent that decreases or increases the expression or activity of the gene products of one or more of ADAM9, MCEMP1, MS4A4A, RAB10, GCLM, EPHX2, RORA, CFAP97, ARL4C, or ACSS1. For example, in some such embodiments, the method comprises (a) sequencing at least part of the subject's genome in a sample from said subject, wherein the at least part of said genome comprises an ADAM9 gene; (b) identifying from the sequencing of said sample at least one single-nucleotide polymorphism (SNP) in ADAM9; and (c) administering a corresponding inhibitor of the ADAM9 gene or its activity.

In other aspects of the invention, disclosed herein are methods of treating or preventing severe COVID-19 in a subject. In some aspects, provided herein are methods for predicting the likelihood of a subject infected with SARS-CoV-2 to progressing to severe COVID-19. In certain embodiments, said methods comprise (a) sequencing and/or measuring (e.g., qPCR, digital PCR) at least part of the subject's transcriptome in a sample from said subject, wherein the at least part of said transcriptome comprises at least one mRNA of ADAM9, MCEMP1, MS4A4A, RAB10, GCLM, EPHX2, RORA, CFAP97, ARL4C, or ACSS1 genes; (b) determining the expression level of at least one of ADAM9, MCEMP1, MS4A4A, RAB10, GCLM, EPHX2, RORA, CFAP97, ARL4C, or ACSS1 of step (a) and comparing it to a reference value, wherein the expression level of at least one of ADAM9, MCEMP1, MS4A4A, RAB10, GCLM, EPHX2, RORA, CFAP97, ARL4C, or ACSS1 gene relative to the reference value indicates whether the subject will respond to a corresponding modulating agent that decreases or increases the expression or activity of the gene products of ADAM9, MCEMP1, MS4A4A, RAB10, GCLM, EPHX2, RORA, CFAP97, ARL4C, and/or ACSS1; and (c) administering said modulating agent that decreases or increases the expression or activity of the gene products of ADAM9, MCEMP1, MS4A4A, RAB10, GCLM, EPHX2, RORA, CFAP97, ARL4C, and/or ACSS1 genes. In some such embodiments, said methods comprise (a) sequencing at least part of the subject's transcriptome in a sample from said subject, wherein the at least part of said transcriptome comprises the mRNA of ADAM9; (b) determining the expression level of the ADAM9 gene at the mRNA or protein level and comparing it to a reference value, wherein the expression level of the ADAM9 gene relative to the reference value indicates whether the subject will respond to an inhibitor of the ADAM9 expression or activity; and (c) administering said modulating agent of ADAM9, MCEMP1, MS4A4A, RAB10, GCLM, EPHX2, RORA, CFAP97, ARL4C, or ACSS1 expression or activity.

In some aspects, provided herein are methods for monitoring a human subject suffering from CoVID-19 for potential treatment with a modulating agent that decreases or increases the expression or activity of the gene products of one or more of ADAM9, MCEMP1, MS4A4A, RAB10, GCLM, EPHX2, RORA, CFAP97, ARL4C, or ACSS1, comprising obtaining a sample from the subject at predetermined intervals. In some embodiments, the methods comprise a) obtaining a gene expression profile from the sample, wherein the expression profile comprises expression levels for one or more genes; wherein said one or more genes comprise at least ADAM9, MCEMP1, MS4A4A, RAB10, GCLM, EPHX2, RORA, CFAP97, ARL4C, or ACSS1; and b) comparing the gene expression profile of each sample chronologically, wherein an increase in one or more of ADAM9, MCEMP1, MS4A4A, RAB10, GCLM, EPHX2, RORA, CFAP97, ARL4C, or ACSS1 expression over time identifies the subject as a critical subject; and c) administering to the subject the corresponding modulating agent or combination of modulating agents. In some preferred embodiments, the methods comprise a) obtaining a gene expression profile from the sample, wherein the expression profile comprises expression levels for ADAM9; and b) comparing the gene expression profile of each sample chronologically, wherein an increase in ADAM9 expression over time identifies the subject as a critical subject; and c) administering to the subject an ADAM9 inhibitor.

Also disclosed herein are methods for predicting the likelihood of a subject infected with SARS-CoV-2 progressing to severe COVID-19. In some embodiments, the methods comprise (a) sequencing or genotyping of at least part of the subject's genome in a sample from said subject, wherein the at least part of said genome comprises one or more of an ADAM9, MCEMP1, MS4A4A, RAB10, GCLM, EPHX2, RORA, CFAP97, ARL4C or ACSS1 gene; (b) identifying from the sequencing or genotyping of said sample at least one SNP in one or more of genes ADAM9, MCEMP1, MS4A4A, RAB10, GCLM, EPHX2, RORA, CFAP97, ARL4C, or ACSS1; and (c) using individual SNPs to form individual SNP risk scores or to combine multiple SNPs to define polygenic risk scores to provide an indication of the likelihood of progression to severe COVID-19.

In some aspects, provided herein are methods for predicting the likelihood of a subject infected with SARS-CoV-2 to progressing to severe COVID-19. In some embodiments, the methods comprise: (a) sequencing or genotyping at least part of the subject's genome in a sample from said subject, wherein the at least part of said genome comprises one or more of an ADAM9, MCEMP1, MS4A4A, RAB10, GCLM, EPHX2, RORA, CFAP97, ARL4C or ACSS1 gene; (b) identifying from the sequencing or genotyping of said sample at least one SNP in one or more of genes ADAM9, MCEMP1, MS4A4A, RAB10, GCLM, EPHX2, RORA, CFAP97, ARL4C, or ACSS1; (c) forming from said at least one SNP a feature vector; and (d) providing the feature vector to a trained classifier and receiving therefrom an indication of the likelihood of progression to severe COVID-19. In some aspects, provided herein are methods for predicting the likelihood of a subject infected with SARS-CoV-2 to progressing to severe COVID-19. In some embodiments, the methods comprise: (a) sequencing or other measurement or measuring (e.g. qPCR, digital PCR) of at least part of the subject's transcriptome in a sample from said subject, wherein the at least part of said transcriptome comprises at least one mRNA of ADAM9, MCEMP1, MS4A4A, RAB10, GCLM, EPHX2, RORA, CFAP97, ARL4C, or ACSS1 genes; (b) determining the expression level of at least one of ADAM9, MCEMP1, MS4A4A, RAB10, GCLM, EPHX2, RORA, CFAP97, ARL4C, or ACSS1 of step (a); (c) forming from said expression level a feature vector; and (d) providing the feature vector to a trained classifier and receiving therefrom an indication of the likelihood of progression to severe COVID-19.

In some aspects, provided herein are methods for predicting the likelihood of a subject infected with SARS-CoV-2 progressing to severe COVID-19, comprising one or more of following steps: (a) measuring the level of soluble ADAM9 protein in a sample from the subject; (b) measuring the expression level of ADAM9 at the RNA level in a sample from the subject; and/or (c) measuring the expression level of ADAM9 at the protein level in a sample from the subject.

In some aspects, provided herein are methods for treating or preventing severe COVID-19 in a subject, comprising measuring in a sample from the subject the expression level of the ADAM9 gene. In some embodiments, measuring the expression level of the ADAM9 gene comprises one or more of: (a) measuring the level of soluble ADAM9 protein; (b) measuring the expression level of ADAM9 at the RNA level; or (c) measuring the expression level of ADAM9 at the protein level; wherein when the level of ADAM9 expression exceeds a threshold limit the subject is administered an ADAM9 inhibitor; and wherein when the level of ADAM9 expression does not exceed said threshold limit the subject is not administered an ADAM9 inhibitor.

In yet further aspects of the invention, provided herein are methods of treating severe COVID-19 in a subject. The disclosed methods of treating severe COVID-19 may include (a) bringing a biological sample into contact with an antibody immobilized on a solid support, wherein said antibody specifically binds an ADAM9-induced peptide cleavage product; (b) incubating the biological sample in contact with the immobilized antibody under conditions such that a cleavage product-antibody complex is formed when the cleaved peptide is present in the biological sample; (c) contacting said cleavage product-antibody complex with a reporter group-conjugated anti-immunoglobulin; (d) incubating the cleavage product-antibody complex in contact with the reporter group-conjugated anti-immunoglobulin under conditions such that a cleavage product-antibody-reporter group-conjugated anti-immunoglobulin complex is formed when the cleaved peptide is present in the biological sample; (e) adding substrate to the cleavage product-antibody-reporter group-conjugated anti-immunoglobulin complex; and (f) measuring a product or a change in the substrate to determine the amount of said cleavage product. In some embodiments, the product or the change in the substrate measured is proportional to the amount of ADAM9-induced peptide cleavage product in the biological sample. In some such embodiments, when the level of ADAM9-induced peptide cleavage product exceeds a threshold limit the subject is administered an ADAM9 inhibitor. In yet further embodiments, when the level of ADAM9-induced peptide cleavage product does not exceed said threshold limit the subject is not administered an ADAM9 inhibitor.

In some aspects, provided herein are methods for predicting the likelihood of a subject infected with SARS-CoV-2 progressing to severe COVID-19 with acute respiratory distress syndrome (ARDS) and initiating treatment. In some embodiments of the invention, the method comprises (a) sequencing of at least part of the subject's transcriptome in a sample from said subject, wherein the at least part of said transcriptome comprises at least the 600 genes in the genomic signature disclosed herein; (b) determining the expression levels of the at least the 600 genes in the genomic signature disclosed herein; (c) forming from said expression levels a feature vector; and (d) providing the feature vector to a trained classifier and receiving therefrom an indication of the likelihood of progression to severe COVID-19.

In some aspects, provided herein are methods for predicting the likelihood of a subject with respiratory symptoms or signs progressing to severe ARDS, and initiating more aggressive or preventative treatment. In some embodiments, the methods comprise (a) sequencing of at least part of the subject's transcriptome in a sample from said subject, wherein the at least part of said transcriptome comprises at least the 600 genes in the genomic signature disclosed herein; (b) determining the expression levels of the at least the 600 genes in the genomic signature disclosed herein; (c) forming from said expression levels a feature vector; and (d) providing the feature vector to a trained classifier and receiving therefrom an indication of the likelihood of progression to severe ARDS.

In certain aspects of the disclosed invention, provided herein are in vitro diagnostic kits for the analysis and/or detection of driver and/or dowstream genes such as (without limitation) one or more of ADAM9, MCEMP1, MS4A4A, RAB10, GCLM, EPHX2, RORA, CFAP97, ARL4C, or ACSS1. In some embodiments, the in vitro diagnostic kits provided herein are for the analysis of at least part of a subjects genome, e.g., for the detection and identification of single-nucleotide polymorphisms (SNPs) in one or more driver and/or dowstream genes disclosed herein. In some embodiments, the in vitro diagnostic kits provided herein are for the detection and/or analysis of the expression level (e.g., transcript or protein level) of one or more driver and/or dowstream genes disclosed herein. For example, and without limitation, such in vitro diagnostic kits contemplated herein are for the detection of protein, such as soluble ADAM9 protein. In some embodiments, the in vitro diagnostic kits provided herein are for the detection and/or analysis of the activity of the gene product of one or more driver and/or dowstream genes disclosed herein, e.g., detection and analysis of the proteolytic activity of ADAM9 protein.

### BRIEF DESCRIPTION OF THE DRAWINGS

FIG. 1 shows the global multi-omics analysis strategy to identify pathways and drivers of ARDS. A. 47 Critical patients (C), 25 Non-critical patients (NC) and 22 Healthy Controls (H) were enrolled in the study. PBMC were isolated by density gradient and frozen in DMSO/FCS until utilization for Helios mass cytometry (Maxpar Direct Immune Profiling System, Fluidigm) and whole proteomics. Plasma was used for cytokine profiling (ELISA for IL-17, V-PLEX Proinflammatory Panel and S-PLEX Human IFN-α2a Kit, Mesoscale Discovery) and whole proteomics. Whole blood was used for RNA-seq (PaxGene tubes, PreAnalytiX) and Whole Genome Sequencing (WGS). The number of treated samples per group and per omics is indicated below each omics' designation. B. RNA-seq pipeline based on NC vs. C comparison. RNA-seq data was split 100 times with 80% for training and the rest for testing. For each partition of the data, feature selection was done based on differential expression; the genes that were significantly differentially expressed for each partition of training data were selected for both the training and corresponding test data. Classification was performed with an ensemble computational approach using 7 different algorithms. After classification and verifying the quality of the results on the test dataset, an ensemble feature ranking score across 6 of the 7 algorithms and all 100 partitions of the data was determined. The top 600 of those features was used as the input for structural causal modeling to derive a putative causal network. C. Cytokines and immune cells were quantified following the manufacturer's instructions. WGS data was used for eQTL analysis together with the gene counts from RNA-seq. Finally, proteomics data were subjected to differential protein expression and nGOseq enrichment analyses. D. The key pathways and drivers resulting from the omics analyses (B and C) were validated in a replication cohort of 81 critical and 73 recovered critical patients. The differential expression of ADAM9, the main driver gene, was compared to publicly available bulk RNA-seq data. Finally, in vitro infection experiments with SARS-CoV-2 were conducted to validate a driver gene candidate.

FIG. 2 shows immune profiling of healthy individuals, non-critical and critical COVID-19 patients: A. Pro-inflammatory cytokines were quantified in plasma by using cytokine profiling assays (V-PLEX Proinflammatory Panel and S-PLEX Human IFN-α2a Kit, Mesoscale Discovery) or ELISA (IL-17, R&D Systems). B. Absolute Lymphocyte counts. Each dot represents a single patient. C. viSNE map colored according to cell density across the three groups. Red indicates the highest density of cells. D-G. Proportions of modified lymphocyte subsets from COVID-19 patients and healthy controls as determined by mass cytometry. Proportions of T-cell subsets (D), B-cell subsets (E), Dendritic cells (F) and Non-classical monocytes (G) are shown. The other cell subsets are presented in FIG. 4. Each dot represents a single patient. In (A) and (D-G), P-values were determined with the Kruskal-Wallis test, followed by Dunn's post-test for multiple group comparison; *P<0.05, **P<0.01, ***P<0.001, ****P<0.0001. In (B), the P-value is determined from a two-tailed unpaired t test; *P<0.05, **P<0.01, ***P<0.001, ****P<0.0001.

FIG. 3 shows Type I interferon response. A. Interferon Stimulated Genes (ISG) scores based on mean normalized expression of six genes (IFI44L, IFI27, RSAD2, SIGLEC1, IFIT1, ISG15) in RNA-seq data. B. Heatmap showing expression of type I IFN-related genes in RNA-seq data. Up-regulated proteins are shown in red and down-regulated proteins are shown in light blue. C. IFNα2a (pg/ml) concentration evaluated by ultra-sensitive S-PLEX Human IFNα2a Kit (Mesoscale Discovery). D. Time-dependent IFNα2a concentration in the critical group. E. Quantification of plasmacytoid dendritic cells as a percentage of PBMCs. P-values were determined with the Kruskal-Wallis test, followed by Dunn's post-test for multiple group comparison; *P<0.05, **P<0.01, ***P<0.001, ****P<0.0001.

FIG. 4 shows immune profiling in healthy individuals, non-critical and critical COVID-19 patients by mass cytometry. Proportions of modified lymphocyte subsets from COVID-19 patients and healthy controls as determined by mass cytometry: proportions of dendritic cells subsets (A), monocytes subsets (B), NK cells subsets (C), NKT (D), γδ T-cells (E) and granulocyte subsets (traces) including neutrophils (F) are shown. Each dot represents a single patient. P-values were determined with the Kruskal-Wallis test, followed by Dunn's post-test for multiple group comparison; *P<0.05, **P<0.01, ***P<0.001, ****P<0.0001.

FIG. 5 shows plasma and PBMC proteomics of healthy individuals, non-critical and critical COVID-19 patients. A. Total number of proteins identified in plasma of patients and healthy controls. Each dot represents a patient. B. Multidimensional scaling plot of normalized intensities of all patients/individuals of the three groups. C. Volcano-plot representing the differentially expressed proteins (DEPs) in Critical versus Non-critical patients. The orange dots represent the proteins that are differentially expressed with a corrected P-value<0.05. Proteins labelled in green and purple represent down-regulated apolipoproteins and up-regulated acute phase proteins, respectively. D. Normalized intensities of the proteins S100A8 and S100A9 in the three groups. P-values were determined with the Kruskal-Wallis test, followed by Dunn's post-test for multiple group comparison; *P<0.05, **P<0.01, ***P<0.001, ****P<0.0001. E. Heatmap showing the expression of apolipoproteins involved in macrophage functions and acute phase proteins in the three groups. Up-regulated proteins are shown in red and down-regulated proteins are shown in light blue. F. Total number of proteins identified in PBMC of patients and healthy controls. Each dot represents a patient. G. Multidimensional scaling plot of normalized intensities of all patients/individuals of the three groups. H. Volcano-plot representing the DEPs in Critical versus Non-critical patients. The orange dots represent the proteins that are differentially expressed with a corrected P-value<0.05. Proteins labelled in green and purple represent up-regulated proteins involved in regulation of blood coagulation and myeloid cell differentiation, respectively. I. Heatmap showing the expression of proteins involved in regulation of blood coagulation and myeloid cell differentiation in the three groups. Up-regulated proteins are shown in red and down-regulated proteins are shown in light blue.

FIG. 6 shows RNA-seq and combined omics analysis of critical patient's specific pathways. A. Volcano plot representing the differentially expressed genes in Critical versus Non-critical patients. The orange dots represent the genes that are differentially expressed with a corrected P-value<0.05. Proteins labeled in green and purple represent up-regulated genes involved in blood pressure regulation and viral entry, respectively. B. Gene set enrichment analysis plots showing positive enrichment of inflammatory response, myeloid leukocyte activation and neutrophil degranulation pathways. NES, normalized enrichment score. C. Enriched nested gene ontology (nGO) categories in critical vs. non-critical patients in RNA-seq, plasma proteomics and PBMC proteomics.

FIG. 7 shows integrated AI/ML and probabilistic programming of non-critical and critical COVID-19 patients. A. ROCs on the train and test set for Critical vs Non-critical groups comparison. All methods perform similarly. Other classification metrics are given in Table 4. B. Putative network showing flow of causal information based on top 600 most informative genes for classifying RNA-seq data of Critical versus Non-critical patients. C. Box plots showing the normalized gene counts of the five driver genes in critical and non-critical patients. The indicated values correspond to the FDR.

FIG. 8 shows results of in silico perturbation experiments. Left: change in BIC (Bayesian Information Criterion) when perturbing each gene individually. Genes are ordered by the change in the number of ancestors minus the number of descendants for the DAG shown in FIG. 7B; i.e., the top 5 driver genes are the 5 leftmost points, and the top 5 response genes are the 5 rightmost points. Right: Change in the BIC of a random sample of 5 genes from the left. The mean BIC of the top 5 driver genes is shown in red.

FIG. 9 shows validation of the RNA-seqsignature-based classification performance of critical and recovered critical COVID-19 patients. A. ROCs on the train and test set for Critical vs Recovered Critical groups comparison in the replication cohort with the 600 gene signature identified from the initial cohort. All methods perform similarly. B. Classification metrics. C. Box plots showing the normalized gene counts of the five driver genes in critical and recovered critical patients. The indicated values correspond to the FDR.

FIG. 10 shows validation of ADAM9 as a key driver for viral infection and replication. A. Quantitative RT-PCR confirmation of differential expression of ADAM9 non-critical vs. critical patients. B. Soluble ADAM9 (sADAM9) concentration in plasma of healthy, non-critical and critical patients determined by ELISA. C. Soluble MICA concentration (sMICA) in serum of healthy, non-critical and critical patients determined by ELISA. D. Expression of ADAM9 according to the genotype of the eQTL rs7840270. E. Experimental approach to assess the viral up-take and the viral replication in silenced Vero-E6 or A549-ACE2 cells. F. Flow-cytometry-based intracellular nucleocapsid staining in control and ADAM9 silenced Vero-E6 and A549-ACE2 cells. G. Quantitative RT-PCR of SARS-CoV-2 in culture supernatant after silencing of ADAM9 in Vero-E6 or A549-ACE2 cells. Results from probe N1 are shown. In (A) and (F-G) the P-value is determined from a two-tailed unpaired t-test; *P<0.05, **P<0.01, ***P<0.001, ****P<0.0001. In (B-D) P-values were determined with the Kruskal-Wallis test, followed by Dunn's post-test for multiple group comparison; *P<0.05, **P<0.01, ***P<0.001, ****P<0.0001.

FIG. 11 shows ADAM9 expression in publicly available data. Box plots showing the normalized gene counts of ADAM9 in healthy (n=17), Severe (n=8) and ICU (n=3) patients in the dataset GSE152418 reported in Arunachalam et al., Science (DOI:10.1126/science.abc6261). The indicated values correspond to the FDR.

FIG. 12 shows validation of ADAM9 silencing. A. Quantitative RT-PCR of the ADAM9 transcript in Vero-E6 or A549-ACE2 cells silenced with a control siRNA or an ADAM9-specific siRNA. The average silencing achieved is 66% and 93% for Vero-E6 and A549-ACE2, respectively (mean of 3 representative experiments). B. Western blot of Vero-E6 and A549-ACE2 cells that have not been transfected (NT), silenced with a control siRNA (ctl) or with an ADAM9-specific siRNA (sil.).

### DETAILED DESCRIPTION

### General

Many studies have reported in great detail the molecular and cellular modifications associated with disease severity, e.g. (Arunachalam et al., 2020; Chua et al., 2020; Hadjadj et al., 2020; Lucas et al., 2020; Messner et al., 2020; Schulte-Schrepping et al., 2020; Shen et al., 2020; Shu et al., 2020; Silvin et al., 2020; Su et al., 2020; Wei et al., 2020; Zhou et al., 2020). But very few have targeted a young population with no or few comorbidities to reduce confounders that also drive severity and mortality; and those were limited to epidemiology and/or standard bio-clinical parameters such as CRP, D-dimers or SOFA scores, e.g. (Ioannidis et al., 2020; Li et al., 2020; Wang et al., 2020). A comprehensive understanding of the immune responses to SARS-CoV-2 infection is fundamental to understand why young patients without comorbidities progress to critical illness and others do not. In particular, knowledge of molecular drivers of critical COVID-19 is urgently needed to identify predictive biomarkers and more efficacious therapeutic targets that work through drivers of severe COVID-19 rather than to secondary reaction genes.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as VH) and a heavy chain constant region. Each light chain includes a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The term “antigen binding site” refers to a region of an antibody or T cell that specifically binds the epitope(s) of an antigen.

The term “binding” or “interacting” refers to an association, which may be a stable association, between two molecules, e.g., between a peptide and a binding partner or agent, e.g., small molecule, due to, for example, electrostatic, hydrophobic, ionic and/or hydrogen-bond interactions under physiological conditions.

The term “biological sample,” “tissue sample,” or simply “sample” includes a tissue sample or a bodily fluid sample. A tissue sample includes, but is not limited to, buccal cells, a brain sample, a skin sample, or an organ sample (e.g., liver). A bodily fluid sample includes all fluids that are present in the body including, but not limited to, blood, plasma, serum, saliva, synovial fluid, lymph, urine, or cerebrospinal fluid. The sample may also be obtained by subjecting it to a pre-treatment step, if necessary, e.g., by homogenizing the sample or by extracting or isolating a component of the sample. Suitable pre-treatment steps may be selected by one skilled in the art depending on nature of the biological sample. One skilled in the art will also appreciate that samples such as serum samples can be diluted prior to analysis. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

“Gene construct”, or simply “construct”, may refer to a nucleic acid, such as a vector, plasmid, viral genome or the like which includes a “coding sequence” for a polypeptide or which is otherwise transcribable to a biologically active RNA (e.g., antisense, decoy, ribozyme, etc.), may be transfected into cells, e.g., mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “polynucleotide”, and “nucleic acid” are used interchangeably. They refer to a natural or synthetic molecule, or some combination thereof, comprising a single nucleotide or two or more nucleotides linked by a phosphate group at the 3′ position of one nucleotide to the 5′ end of another nucleotide. The polymeric form of nucleotides is not limited by length and can comprise either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides. The polynucleotide is not necessarily associated with the cell in which the nucleic acid is found in nature, and/or operably linked to a polynucleotide to which it is linked in nature.

The terms “protein”, “peptide”, “polypeptide” and “polypeptide fragment” may be used interchangeably herein to refer to polymers of amino acid, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The terms “polypeptidefragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In other embodiments, a fragment may have immunogenic properties.

As used herein, “specific binding” refers to the ability of an antibody to bind to a predetermined antigen or the ability of a peptide to bind to its predetermined binding partner. Typically, an antibody or peptide specifically binds to its predetermined antigen or binding partner with an affinity corresponding to a KD of about 10-7 M or less, and binds to the predetermined antigen/binding partner with an affinity (as expressed by KD) that is at least 10 fold less, at least 100 fold less or at least 1000 fold less than its affinity for binding to a non-specific and unrelated antigen/binding partner (e.g., BSA, casein).

The term “specifically binds” or “specific binding”, as used herein, when referring to a polypeptide (including antibodies) or receptor, may refer to a binding reaction which is determinative of the presence of the protein or polypeptide or receptor in a heterogeneous population of proteins and other biologics; or to a binding reaction that results in blocking and/or inhibiting the expression and/or activity of a target gene. Thus, under designated conditions (e.g., immunoassay conditions in the case of an antibody), a specified ligand or antibody “specifically binds” to its particular “target” (e.g., an antibody specifically binds to an antigen) when it does not bind in a significant amount to other proteins present in the sample or to other proteins to which the ligand or antibody may come in contact in an organism. Generally and without being bond by theory, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10¹ M⁻¹, 10¹⁰ M⁻¹, 10¹¹ M⁻¹, and 10¹² M⁻¹ or more) with that second molecule.

As used herein, the term “subject” means a human or non-human animal selected for treatment or therapy.

The terms “transformation”, “transfection”, or “transduction” mean the introduction of a nucleic acid, e.g., an expression vector, into a recipient cell (e.g., a mammalian cell) including introduction of a nucleic acid to the chromosomal DNA of said cell.

The term “immunogenic or antigenic polypeptide” as used herein includes polypeptides that are immunologically active in the sense that once administered to the host or a sample from said host, it is able to evoke an immune response of the humoral and/or cellular type directed against the protein (e.g., the binding of antibodies to the antigenic peptide, such as neutralizing antibodis). An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. As discussed herein, the invention encompasses active fragments and variants of the antigenic polypeptide. Preferably the protein fragment is such that it has substantially the same immunological activity as the total protein. Thus, a protein fragment according to the invention comprises or consists essentially of or consists of at least one epitope or antigenic determinant. Thus, the term “immunogenic or antigenic peptide/polypeptide” further contemplates deletions, additions and substitutions to the sequence, so long as the polypeptide functions to produce an immunological response as defined herein. Such includes amino acid or peptide sequence having conservative amino acid substitutions, non-conservative amino acid substitutions (e.g., a degenerate variant), substitutions within the wobble position of each codon (e.g., DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, to which the nucleic acid has been linked, and may or may not be able to replicate autonomously or integrate into a chromosome of a host cell. Such vectors may include any vector, (e.g., a plasmid, cosmid or phage chromosome) containing a gene construct in a form suitable for expression by a cell (e.g., linked to a transcriptional control element).

In some aspects of the disclosed invention, provided herein are methods for treating or preventing severe coronavirus disease 2019 (COVID-19) in a subject, comprising administering to the subject a composition comprising a modulating agent of ADAM9, MCEMP1, MS4A4A, RAB10, GCLM, EPHX2, RORA, CFAP97, ARL4C, ACSS1, or any combination thereof. The modulating agents contemplated and disclosed herein may decrease or increase the activity or level of the corresponding gene products (e.g., transcript and/or protein). Preferably, the compositions disclosed herein comprise at least an inhibitor of ADAM9.

In some aspects of the invention, provided herein are methods of treating and/or preventing severe COVID-19 in a subject. In further aspects, provided herein are methods for predicting the likelihood of a subject infected with SARS-CoV-2 progressing to severe COVID-19. In some embodiments, such methods include (a) sequencing at least part of the subject's genome in a sample from said subject, wherein the at least part of said genome comprises an ADAM9, MCEMP1, MS4A4A, RAB10, GCLM, EPHX2, RORA, CFAP97, ARL4C, or ACSS1 gene; (b) identifying from the sequencing of said sample at least one at least one single-nucleotide polymorphism (SNP) in one or more of genes: ADAM9, MCEMP1, MS4A4A, RAB10, GCLM, EPHX2, RORA, CFAP97, ARL4C, or ACSS1; and (c) administering a corresponding modulating agent that decreases or increases the expression or activity of the gene products of one or more of ADAM9, MCEMP1, MS4A4A, RAB10, GCLM, EPHX2, RORA, CFAP97, ARL4C, or ACSS1. For example, in some such embodiments, the method comprises (a) sequencing at least part of the subject's genome in a sample from said subject, wherein the at least part of said genome comprises an ADAM9 gene; (b) identifying from the sequencing of said sample at least one single-nucleotide polymorphism (SNP) in ADAM9; and (c) administering a corresponding inhibitor of the ADAM9 gene or its activity.

In some embodiments, the consequence of the at least one SNP is a frameshift mutation, nonsense mutation, missense mutation, or splice-site variant mutation. In some embodiments, the at least one SNP is located in a non-coding region of the gene and/or corresponding mRNA transcript. In some such embodiments, the consequence of the at least one SNP is a 5′ UTR variant, a 3′ UTR variant, or an intron variant. For example, and without limitation, such SNPs include rs7840270, rs7831735, rs11465401, rs11465397, rs189755275, rs76847438, rs10736707, and rs10792287. Preferably, the SNPs of interest are rs7840270 and/or rs7831735.

In other aspects of the invention, disclosed herein are methods of treating and/or preventing severe COVID-19 in a subject. In some aspects, provided herein are methods for predicting the likelihood of a subject infected with SARS-CoV-2 progressing to severe COVID-19 (i.e., a critical COVID-19 subject). In certain embodiments, said methods comprise (a) sequencing at least part of the subject's transcriptome in a sample from said subject, wherein the at least part of said transcriptome comprises at least one mRNA of ADAM9, MCEMP1, MS4A4A, RAB10, GCLM, EPHX2, RORA, CFAP97, ARL4C, or ACSS1; (b) determining the expression level of at least one of ADAM9, MCEMP1, MS4A4A, RAB10, GCLM, EPHX2, RORA, CFAP97, ARL4C, or ACSS1 of step (a) and comparing it to a reference value, wherein the expression level of at least one of ADAM9, MCEMP1, MS4A4A, RAB10, GCLM, EPHX2, RORA, CFAP97, ARL4C, or ACSS1 gene relative to the reference value indicates whether the subject will respond to a corresponding modulating agent that decreases or increases the expression or activity of the gene products of ADAM9, MCEMP1, MS4A4A, RAB10, GCLM, EPHX2, RORA, CFAP97, ARL4C, and/or ACSS1 genes; and (c) administering said modulating agent that decreases or increases the expression or activity of the gene products of ADAM9, MCEMP1, MS4A4A, RAB10, GCLM, EPHX2, RORA, CFAP97, ARL4C, and/or ACSS1 to the subject. In some such embodiments, said methods comprise (a) sequencing at least part of the subject's transcriptome in a sample from said subject, wherein the at least part of said transcriptome comprises the mRNA of ADAM9; (b) determining the expression level of the ADAM9 gene at the mRNA or protein level and comparing it to a reference value, wherein the expression level of the ADAM9 gene relative to the reference value indicates whether the subject will respond to an inhibitor of the ADAM9 expression or activity; and (c) administering said inhibitor of ADAM9 to the subject.

In some embodiments the expression level reference value is derived from a sample from a non-critical subject suffering from COVID-19 or is indicative of a non-critical subject suffering from COVID-19. Thus, in some embodiments, the expression level reference value is derived from a sample from an asymptomatic subject infected with SARS-CoV-2 or is indicative of an asymptomatic subject infected with SARS-CoV-2. In other embodiments, the expression level reference value is derived from a sample from a healthy subject or is indicative of a healthy subject.

In some aspects, provided herein are methods for monitoring a human subject suffering from CoVID-19 for potential treatment with a modulating agent that decreases or increases the expression or activity of the gene products of one or more of ADAM9, MCEMP1, MS4A4A, RAB10, GCLM, EPHX2, RORA, CFAP97, ARL4C, or ACSS1, comprising obtaining a sample from the subject at predetermined intervals. In some embodiments, the methods comprise a) obtaining a gene expression profile from the sample, wherein the expression profile comprises expression levels for one or more genes; wherein said one or more genes comprise one or more of ADAM9, MCEMP1, MS4A4A, RAB10, GCLM, EPHX2, RORA, CFAP97, ARL4C, or ACSS1; and b) comparing the gene expression profile of each sample chronologically, wherein an increase in one or more of ADAM9, MCEMP1, MS4A4A, RAB10, GCLM, EPHX2, RORA, CFAP97, ARL4C, or ACSS1 expression over time identifies the subject as a critical subject; and c) administering to the subject the corresponding modulating agent or combination of modulating agents. In some preferred embodiments, the methods comprise a) obtaining a gene expression profile from the sample, wherein the expression profile comprises expression levels for ADAM9; and b) comparing the gene expression profile of each sample chronologically, wherein an increase in ADAM9 expression over time identifies the subject as a critical subject; and c) administering to the subject an ADAM9 inhibitor.

Also disclosed herein are methods for predicting the likelihood of a subject infected with SARS-CoV-2 progressing to severe COVID-19. In some embodiments, the methods comprise (a) sequencing or genotyping of at least part of the subject's genome in a sample from said subject, wherein the at least part of said genome comprises one or more of an ADAM9, MCEMP1, MS4A4A, RAB10, GCLM, EPHX2, RORA, CFAP97, ARL4C or ACSS1 gene; (b) identifying from the sequencing or genotyping of said sample at least one SNP in one or more of genes ADAM9, MCEMP1, MS4A4A, RAB10, GCLM, EPHX2, RORA, CFAP97, ARL4C, or ACSS1; and (c) using individual SNPs to form individual SNP risk scores or to combine multiple SNPs to define polygenic risk scores to provide an indication of the likelihood of progression to severe COVID-19.

In some aspects, provided herein are methods for predicting the likelihood of a subject infected with SARS-CoV-2 to progressing to severe COVID-19. In some embodiments, the methods comprise: (a) sequencing or genotyping at least part of the subject's genome in a sample from said subject, wherein the at least part of said genome comprises one or more of an ADAM9, MCEMP1, MS4A4A, RAB10, GCLM, EPHX2, RORA, CFAP97, ARL4C or ACSS1 gene; (b) identifying from the sequencing or genotyping of said sample at least one SNP in one or more of genes ADAM9, MCEMP1, MS4A4A, RAB10, GCLM, EPHX2, RORA, CFAP97, ARL4C, or ACSS1; (c) forming from said at least one SNP a feature vector; and (d) providing the feature vector to a trained classifier and receiving therefrom an indication of the likelihood of progression to severe COVID-19. In some aspects, provided herein are methods for predicting the likelihood of a subject infected with SARS-CoV-2 to progressing to severe COVID-19. In some embodiments, the methods comprise: (a) sequencing or other measurement or measuring (e.g. qPCR, digital PCR) of at least part of the subject's transcriptome in a sample from said subject, wherein the at least part of said transcriptome comprises at least one mRNA of ADAM9, MCEMP1, MS4A4A, RAB10, GCLM, EPHX2, RORA, CFAP97, ARL4C, or ACSS1 genes; (b) determining the expression level of at least one of ADAM9, MCEMP1, MS4A4A, RAB10, GCLM, EPHX2, RORA, CFAP97, ARL4C, or ACSS1 of step (a); (c) forming from said expression level a feature vector; and (d) providing the feature vector to a trained classifier and receiving therefrom an indication of the likelihood of progression to severe COVID-19.

In some embodiments, the trained classifier comprises a LASSO model, a ridge regression model, a support vector machine (SVM), a quantum support vector machine (qSVM), an XGBoost model (XGB) a random forest (RF), or a DANN artificial neural network.

In some aspects, provided herein are methods for predicting the likelihood of a subject infected with SARS-CoV-2 progressing to severe COVID-19, comprising one or more of following steps: (a) measuring the level of soluble ADAM9 protein in a sample from the subject; (b) measuring the expression level of ADAM9 at the RNA level in a sample from the subject; and/or (c) measuring the expression level of ADAM9 at the protein level in a sample from the subject.

In some aspects, provided herein are methods for treating or preventing severe COVID-19 in a subject, comprising measuring in a sample from the subject the expression level of the ADAM9 gene. In some embodiments, measuring the expression level of the ADAM9 gene comprises one or more of: (a) measuring the level of soluble ADAM9 protein; (b) measuring the expression level of ADAM9 at the RNA level; or (c) measuring the expression level of ADAM9 at the protein level; wherein when the level of ADAM9 expression exceeds a threshold limit the subject is administered an ADAM9 inhibitor; and wherein when the level of ADAM9 expression does not exceed said threshold limit the subject is not administered an ADAM9 inhibitor.

In yet further aspects of the invention, provided herein are methods of treating severe COVID-19 in a subject. The disclosed methods of treating severe COVID-19 may include (a) bringing a biological sample into contact with an antibody immobilized on a solid support, wherein said antibody specifically binds an ADAM9-induced peptide cleavage product; (b) incubating the biological sample in contact with the immobilized antibody under conditions such that a cleavage product-antibody complex is formed when the cleaved peptide is present in the biological sample; (c) contacting said cleavage product-antibody complex with a reporter group-conjugated anti-immunoglobulin; (d) incubating the cleavage product-antibody complex in contact with the reporter group-conjugated anti-immunoglobulin under conditions such that a cleavage product-antibody-reporter group-conjugated anti-immunoglobulin complex is formed when the cleaved peptide is present in the biological sample; (e) adding substrate to the cleavage product-antibody-reporter group-conjugated anti-immunoglobulin complex; and (f) measuring a product or a change in the substrate to determine the amount of said cleavage product. In some embodiments, the product or the change in the substrate measured is proportional to the amount of ADAM9-induced peptide cleavage product in the biological sample. In some such embodiments, when the level of ADAM9-induced peptide cleavage product exceeds a threshold limit the subject is administered an ADAM9 inhibitor. In yet further embodiments, when the level of ADAM9-induced peptide cleavage product does not exceed said threshold limit the subject is not administered an ADAM9 inhibitor.

In some aspects, provided herein are methods for predicting the likelihood of a subject infected with SARS-CoV-2 progressing to severe COVID-19 with acute respiratory distress syndrome (ARDS) and initiating treatment. In some embodiments of the invention, the method comprises (a) sequencing of at least part of the subject's transcriptome in a sample from said subject, wherein the at least part of said transcriptome comprises at least the 600 genes in the genomic signature disclosed herein; (b) determining the expression levels of the at least the 600 genes in the genomic signature disclosed herein; (c) forming from said expression levels a feature vector; and (d) providing the feature vector to a trained classifier and receiving therefrom an indication of the likelihood of progression to severe COVID-19.

In some aspects, provided herein are methods for predicting the likelihood of a subject with respiratory symptoms or signs progressing to severe ARDS, and initiating more aggressive or preventative treatment. In some embodiments, the methods comprise (a) sequencing of at least part of the subject's transcriptome in a sample from said subject, wherein the at least part of said transcriptome comprises at least the 600 genes in the genomic signature disclosed herein; (b) determining the expression levels of the at least the 600 genes in the genomic signature disclosed herein; (c) forming from said expression levels a feature vector; and (d) providing the feature vector to a trained classifier and receiving therefrom an indication of the likelihood of progression to severe ARDS.

In addition to SARS-CoV-2 infection (and COVID-19 disease) those of skill in the art will appreciate that ARDS also typically occurs in people who are already critically ill or who have significant injuries. The signs and symptoms of ARDS can vary in intensity and can include, Severe shortness of breath, labored and unusually rapid breathing, low blood pressure, confusion and extreme tiredness. The underlying causes of ARDS may include sepsis; damage to the tissues of the lungs such as by inhalation of harmful substances (e.g., high concentrations of smoke, chemical fumes/inhalants, as well as damage caused by aspiration, such as the aspiration of vomit or as a result near-drowning; severe pneumonia, physical traumatic such as to the head, chest, or other major injury (e.g., damage caused by falls, car crashes, gunshot wounds, and the like); pancreatitis; severe burn injury; massive blood transfusion. Accordingly, in some embodiments, the subject is suffering from a viral infection. In other embodiments the subject is suffering from a non-viral infection or inflammation. In some embodiments, the subject is suffering from traumatic injury.

In some embodiments, the sample is a tissue sample or a bodily fluid sample. Preferably, the sample is a blood sample. In some embodiments, the sample comprises serum or sera derived from the subject.

### Therapeutic Methods

The treatment approaches disclosed herein take advantage of an advanced integrated machine learning and probabilistic programming strategy for high-resolution molecular analyses of well-defined cohorts of patients. The investigation of causal molecular drivers of severe forms of COVID-19 in small, tightly controlled patient cohorts lead to the discovery that certain driver genes may be responsible for the development of critical illness, and may represent therapeutic targets. Thus, disclosed herein are agents (e.g., activators and/or inhibitors) that modulate the activity and/or the expression of a target gene (e.g., the level of transcript or active protein).

Without being bound by any particular theory, such agents include modulating agents of ADAM9, MCEMP1, MS4A4A, RAB10, GCLM, EPHX2, RORA, CFAP97, ARL4C, and/or ACSS1. In some embodiments, the modulating agent is a chemical compound, a small molecule, a mixture of chemical compounds and/or a biological macromolecule (such as a nucleic acid, an antibody, an antibody fragment, a protein or a peptide). Moreover, the agents contemplated herein include those disclosed herein, those known in the art, and those that may be identified by screening or validation assays disclosed herein.

In some embodiments, the modulating agent is an inhibitor. Preferably, the agent is an inhibitor of ADAM9. Small molecule inhibitors known in the art include Batimastat, Marimastat, and CGS27023.

In some embodiments, the the modulating agent is an antibody or antibody fragment that binds specifically to the protein expressed by the target gene. In some embodiments, the antibody depletes, neutralizes, or inhibits one or more associated activities of said protein. Such antibodies include, but are not limited to, RAV-18, KID-24, and fragments thereof. On the other hand, the antibody may induce/activate or enhance one or more associated activities of said protein, such as anti-CD79b and the like.

In some embodiments, the inhibitor is an interfering nucleic acid specific for an mRNA product of a target gene disclosed herein. Such interfering nucleic acids are known in the art and include, without limitation, siRNAs, shRNAs, miRNAs, peptide nucleic acids (PNAs), and the like, as are known in the art. Preferably, the interfering nucleic acid is a siRNA, such as HSS112867 (Thermofisher Scientific, US).

It will be appreciated by those of skill in the relevant art that a personalized medicine (e.g., a personalized therapeutic composition and/or therapeutic regimen) may be administered to a human subject. For example, without being bound by any particular theory or methodology, a combination of modulating agents may be administered to the subject in need thereof. In such embodiments, the combination and administration of such modulating agents is informed, at least in part, by the methods disclosed herein. In some embodiments, the combination of modulating agents may be of inhibitors or activators of a plurality of different genes, multiple inhibitors or activators of the same gene, or combinations of such inhibitors and activators. In some such embodiments, the combination of modulatory agents can be administered either in the same formulation or in separate formulations, either concomitantly or sequentially. Thus, a subject who receives such personalized treatment can benefit from a combined effect of different therapeutic agents.

Also contemplated herein are kits for use in performing any of the methods disclosed herein.

### Kits and Diagnostic Systems

A diagnostic system of the invention disclosed herein may be in the form of a kit. Such kits as are contemplated herein include, in sufficient for at least one assay, a composition comprising a coronavirus antigen of the current invention as a separately packaged reagent. Instructions for use of the packaged reagent are also typically included. “Instructions for use” typically include a tangible expression describing the reagent concentration or at least one assay method parameter such as the relative amounts of reagent and sample to be admixed, maintenance time periods for reagent/sample admixtures, temperature, buffer conditions and the like. Thus, provided herein are in vitro diagnostic kits for the analysis and/or detection of driver and/or dowstream genes such as (without limitation) one or more of ADAM9, MCEMP1, MS4A4A, RAB10, GCLM, EPHX2, RORA, CFAP97, ARL4C, or ACSS1. In some embodiments, the in vitro diagnostic kits provided herein are for the analysis of at least part of a subject's genome, e.g., for the detection and identification of single-nucleotide polymorphisms (SNPs) in one or more and/or dowstream genes disclosed herein. In some embodiments, the in vitro diagnostic kits provided herein are for the detection and/or analysis of the expression level (e.g., transcript or protein level) of one or more and/or dowstream genes disclosed herein. For example, and without limitation, such in vitro diagnostic kits contemplated herein are for the detection of soluble ADAM9 protein. In some embodiments, the in vitro diagnostic kits provided herein are for the detection and/or analysis of the activity of the gene product of one or more and/or dowstream genes disclosed herein, e.g., detection and analysis of the proteolytic activity of ADAM9 protein.

In preferred embodiments, the diagnostic system of the present invention further includes a label or indicating means capable of signaling the formation of a complex containing a recombinant antigen. As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in an expressed protein or polypeptide, or used separately, and those atoms or molecules can be used alone or in conjunction with additional reagents. Such labels are themselves well-known in clinical diagnostic chemistry and constitute a part of this invention only insofar as they are utilized with otherwise novel proteins methods and/or systems.

As a non-limiting example, the diagnostic kits of the present invention can be used in an “ELISA” format to detect and quantify peptides, proteins, antibodies, and hormones of interest identified by the methods disclosed herein. Generally, “ELISA” refers to an enzyme-linked immunosorbent assay that employs an antibody or antigen bound to a solid phase and an enzyme-antigen or enzyme-antibody conjugate to detect and quantify the amount of an antigen or antibody present in a sample. A description of the ELISA technique is found in Chapter 22 of the 4th Edition of Basic and Clinical Immunology by D. P. Sites et al., published by Lange Medical Publications of Los Altos, Calif. in 1982 and in U.S. Pat. Nos. 3,654,090; 3,850,752; and 4,016,043, which are all incorporated herein by reference.

### Exemplification

### Example 1: Materials and Methods

### Patients

Patients aged under 50 years of old, without major comorbidities, admitted for COVID-19 in the infectious disease unit (hereafter designated non-critical care ward) or at designated intensive care units (ICUs) of a university hospital network in northeast France (Alsace, France) were investigated within the framework of the present study. Among comorbidities, only hypertension and obesity were not an exclusion criteria. Follow-up was performed until hospital discharge. SARS-CoV-2 infection was confirmed in all patients by quantitative real-time reverse transcriptase PCR tests for COVID-19 nucleic acid on nasopharyngeal swabs in accordance with WHO-defined protocol (www.who.int/docs/default-source/coronaviruse/real-time-rt-pcr-assays-for-the-detection-of-sars-cov-2-institut-pasteur-paris.pdf). Patients were managed following the current guidelines at the time (Alhazzani et al., 2020), without specific therapeutic intervention.

Three groups were considered:
(1) the “critical group” including 47 patients admitted to intensive care unit (ICU) and patients who were transferred from ward to ICU,
(2) the “non-critical group” composed of 25 hospitalized patients in the medicine ward,
(3) the “healthy control group” including 22 healthy age and sex-matched blood donors under 50 years old were included as a “control group”.

Blood sampling was performed at ward/ICU admission and for ICU patients every four days until hospital discharge.

A replication cohort composed of 81 critical patients and 73 recovered critical patients from one of the ICU departments of Strasbourg University hospitals was used to validate molecular findings.

### Sampling

Venipunctures were performed at admission in ICU or medical ward within the framework or routine diagnostic procedures. A subset of ICU patients (73%) were sampled every 4-8 days post-hospitalization until discharge or death. Patient blood was collected in a BD Vacutainer tube with Heparin (for plasma and PBMC), EDTA (for DNA) or without additive (for serum) and in PAXgene® Blood RNA tubes (Becton, Dickinson and Company, USA). Healthy donors were sampled in BD Vacutainer tubes with Heparin, with EDTA or without additive. Plasma and serum fractions were collected after centrifugation at 1200×g at room temperature for 10 min, aliquoted, and stored at −80° C. until use. Peripheral Blood Mononuclear Cells (PBMCs) were prepared within 24 h by Ficoll density gradient. Aliquots of 1×10⁶ cells dry cell pellets were frozen at −80° C. until their use for proteomics. Aliquots of a minimum of 5×10⁶ cells were frozen at −80° C. in 80% fetal calf serum (FCS)/20% Dimethyl Sulfoxide (DMSO). EDTA and PAXgene® tubes were stored at −80° C. until use for DNA and RNA extraction, respectively.

### Cytokine Profiling

Plasma were analyzed with the V-PLEX Proinflammatory Panel 1 Human Kit (IL-6, IL-8, IL-10, TNF-α, IL-12p70, IL-1β, GM-CSF, IL-2, and IFN-γ) and the S-PLEX Human IFN-α2a Kit following the manufacturer's instructions (Mesoscale Discovery, USA). Plasma were used undiluted for the S-PLEX Human IFN-α2a Kit and diluted 2 times for the V-PLEX Proinflammatory Panel 1. MSD plates were analyzed on the MS2400 imager (Mesoscale Discovery, Gaithersburg, MD). Soluble IL-17 was quantified by Quantikine® HS ELISA (Human IL-17 Immunoassay) on undiluted serum followings the manufacturer's instructions (R&D Systems, Minneapolis, MN). All standards and samples were measured in duplicate.

### Immune Phenotyping by Mass Cytometry

PBMC were thawed rapidly and washed twice with 10 volumes of RPMI (Roswell Park Memorial Institute) medium (ThermoFisher Scientific, USA) and centrifuged 7 min at 300×g at room temperature between each washing step. Cells were then treated with 250 U of DNAse (ThermoFisher Scientific, USA) in 10 volumes of RPMI medium during 30 min at 37° C./5% CO₂. During this step, the viability and the numeration of cells was performed with Trypan Blue (ThermoFisher Scientific, USA) and TUrk solution (Merck Millipore, USA), respectively. After elimination of the DNAse by centrifugation during 7 min at 300×g at room temperature, a total of 3×10⁶ cells were used for immunostaining with the Maxpar® Direct Immune Profiling Assay kit (Fluidigm, USA), following the manufacturer's instructions. Prepared cells were stored at −80° C. until their use for acquisition on the Helios mass cytometer system. An average of 600,000 events were acquired per sample. Mass cytometry standard files produced by the HELIOS were analyzed using Maxpar® Pathsetter software v.2.0.45 that was modified for the live/dead parameters: tallest peak was selected instead of closest peak for the identification and quantification of the cell populations. FCS files of each group (Healthy, Critical, Non-Critical) were then concatenated with CyTOF® software v.7.0.8493.0 for viSNE analysis (Cytobank Inc, USA). A total of 300,000 events were used for viSNE maps that was generated with the following parameters: iterations (1,000), perplexity (30) and theta (0.5). ViSNE maps are presented as means of all samples in each group.

### Plasma Proteomics Analysis

### Sample Preparation

Samples were prepared using the PreOmics iST Kit (PreOmics GmbH, Martinsried, Germany) according to the manufacturer's protocol. Two μl of plasma were mixed with 50 μl Lyse buffer. Briefly, protein concentration was determined using the Bradford assay (Biorad, USA) according to the manufacturer's instructions. Samples were transferred to 96 well-plate cartridges. Then, 50 μl of resuspended Digest solution were added and samples were heated at 37° C. for 2 h before adding 100 μl of Stop buffer. Samples were centrifuged in order to retain the peptides on the cartridge and washed twice with “Wash 1” and “Wash 2” buffers. Peptides were then eluted twice with Elute buffer before evaporation under vacuum. Finally, peptides were resuspended using the “LC-load” solution containing iRT peptides (Biognosys, Zurich, Switzerland) and samples were quickly sonicated before being analyzed.

### NanoLC-MS/MS Analysis

NanoLC-MS/MS analyses were performed on a nanoAcquity UltraPerformance LC® (UPLC®) device (Waters Corporation, USA) coupled to a Q-Exactive™ Plus mass spectrometer (Thermo Fisher Scientific, USA). Peptide separation was performed on an ACQUITY UPLC BEH130 C18 column (250 mm×75 μm with 1.7 μm diameter particles) and a Symmetry C18 precolumn (20 mm×180 μm with 5 μm diameter particles, Waters). The solvent system consisted of 0.1% FA in water (solvent A) and 0.1% FA in ACN (solvent B). Samples (equivalent to 500 ng of proteins) were loaded into the enrichment column over 3 min at 5 μL/min with 99% of solvent A and 1% of solvent B. The peptides were eluted at 400 nL/min with the following gradient of solvent B: from 1 to 35% over 60 min and 35 to 90% over 1 min. The 93 samples were injected in randomized order. The MS capillary voltage was set to 2.1 kV at 250° C. The system was operated in Data Dependent Acquisition mode with automatic switching between MS (mass range 300-1800 m/z with R=70,000, Automatic gain control (AGC) fixed at 3×106 ions and a maximum injection time set at 50 ms) and MS/MS (mass range 200-2000 m/z with R=17,500, AGC fixed at 1×105 and the maximal injection time set to 100 ms) modes. The ten most abundant ions were selected on each MS spectrum for further isolation and higher energy collision dissociation fragmentation, excluding unassigned and monocharged ions. The dynamic exclusion time was set to 60 s. A sample pool comprising equal amounts of all protein extracts was constituted and regularly injected during the course of the experiment, as an additional Quality Control.

### Data Analysis

Raw data obtained for each sample (45 Critical patients, 23 Non-critical patients, and 22 Healthy controls) were processed using MaxQuant software (version 1.6.14). Peaks were assigned with the Andromeda search engine with trypsin/P specificity. A database containing all human entries was extracted from UniProtKB-SwissProt database (as of May 11, 2020; 20410 entries). The minimal peptide length required was seven amino acids and a maximum of one missed cleavage was allowed. Methionine oxidation and acetylation of protein's N-termini were set as variable modifications and acetylated and modified methionine-containing peptides, as well as their unmodified counterparts, were excluded from protein quantification. Cysteine carbamidomethylation was set as a fixed modification. For protein quantification, the “match between runs” option was enabled. The maximum false discovery rate was set to 1% at peptide and protein levels with the use of a decoy strategy. LFQ intensities were extracted from the ProteinGroups.txt file after removal of non-human and keratin contaminants, as well as reverse and proteins only identified by site. Complete datasets have been deposited in the ProteomeXchange Consortium database with the identifier PXD 025265 (Alhazzani et al., 2020).

### Differential Protein Expression Analysis

Normalized label-free quantification (LFQ) values from MaxQuant software were used for differential protein expression analysis. For each pairwise comparison, proteins expressed in at least 80% of the samples in either group were retained. Variance stabilization normalization (Vsn) was performed using justvsn function from the vsn R package (Huber et al., 2002). Missing values were imputed using the Random Forest approach (Kokla et al., 2019). This resulted in 161 proteins. Differential protein expression analysis was performed using limma bioconductor package in R (Ritchie et al., 2015). Significant differentially expressed proteins were determined based on an adjusted p-value cut-off of 0.05 using the Benjamini-Hochberg method.

### PBMC Proteomics Analysis

Samples were prepared using the PreOmics' iST Kit (PreOmics GmbH, Martinsried, Germany) according to the manufacturer's protocol. Briefly, PBMC pellets were resuspended in 50 μl Lyse buffer and heated at 95° C. for 10 min at 1,000 rpm before being sonicated for 10 min on ice. Protein concentration of the extract was determined using the Bradford assay (Biorad, Hercules, USA) according to the manufacturer's instructions. Samples were transferred to 96 well-plate cartridges. Then, 50 μl of resuspended Digest solution were added and samples were heated at 37° C. for 2 h before adding 100 μl of Stop buffer. Samples were centrifuged in order to retain the peptides on the cartridge and washed twice with “Wash 1” and “Wash 2” buffers. Peptides were then eluted twice with Elute buffer before evaporation under vacuum. Finally, peptides were resuspended using the “LC-load” solution containing iRT peptides (Biognosys, Switzerland) and samples were quickly sonicated before being analyzed.

### NanoLC-MS/MS Analysis

NanoLC-MS/MS analyses were performed on a nanoAcquity UPLC device (Waters Corporation, USA) coupled to a Q-Exactive HF-X mass spectrometer (Thermo Fisher Scientific, USA). Peptide separation was performed on an Acquity UPLC BEH130 C18 column (250 mm×75 μm with 1.7 μm diameter particles) and a Symmetry C18 precolumn (20 mm×180 μm with 5 μm diameter particles, Waters). The solvent system consisted of 0.1% Formic Acid (FA) in water (solvent A) and 0.1% FA in Acetonitrile (ACN) (solvent B). Samples (equivalent to 414 ng of proteins) were loaded into the enrichment column over 3 min at 5 μL/min with 99% of solvent A and 1% of solvent B. The peptides were eluted at 400 nL/min with the following gradient of solvent B: from 2 to 25% over 53 min, 25 to 40% over 10 min and 40 to 90% over 2 min. The 77 samples were injected using a randomized injection sequence. The MS capillary voltage was set to 1.9 kV at 250° C. The system was operated in Data Dependent Acquisition mode with automatic switching between MS (mass range 300-1800 m/z with R=60,000, Automatic gain control (AGC) fixed at 3×10⁶ ions and a maximum injection time set at 50 ms) and MS/MS (mass range 200-2000 m/z with R=15,000, AGC fixed at 1×10⁵ and the maximal injection time set to 100 ms) modes. The ten most abundant ions were selected on each MS spectrum for further isolation and higher energy collision dissociation fragmentation, excluding unassigned and monocharged ions. The dynamic exclusion time was set to 60 s. A sample pool comprising equal amounts of all protein extracts was constituted and regularly injected during the course of the experiment, as an additional Quality Control.

### Data Analysis

Raw data obtained for each sample (34 Critical Patients, 21 Non-Critical patients and 22 healthy controls) were processed using MaxQuant software (version 1.6.14). Peaks were assigned with the Andromeda search engine with trypsin/P specificity. A combined human and bovine database (because of potential traces of fetal calf serum in samples) was extracted from UniProtKB-SwissProt (as of Sep. 8, 2020, 26,413 entries). The minimal peptide length required was seven amino acids and a maximum of one missed cleavage was allowed. Methionine oxidation and acetylation of protein's N-termini were set as variable modifications and acetylated and modified methionine-containing peptides, as well as their unmodified counterparts, were excluded from protein quantification. Cysteine carbamidomethylation was set as a fixed modification. For protein quantification, the “match between runs” option was enabled. The maximum false discovery rate was set to 1% at peptide and protein levels with the use of a decoy strategy. Only peptides unique to human entries were kept and their intensities were summed to derive protein intensities. Complete datasets have been deposited in the ProteomeXchange Consortium database with the identifier PXD 025265 (Deutsch et al., 2017).

### Differential Protein Expression Analysis

Normalized label-free quantification (LFQ) values from MaxQuant software were used for differential protein expression analysis. For each pairwise comparison, proteins expressed in at least 80% of the samples in either group were retained. Variance stabilization normalization (Vsn) was performed using justvsn function from the vsn R package (Huber et al., 2002). Missing values were imputed using the Random Forest approach (Kokla et al., 2019). This resulted in 732 proteins. Differential protein expression analysis was performed using limma bioconductor package in R (Ritchie et al., 2015). Significant differentially expressed proteins were determined based on an adjusted p-value cut-off of 0.05 using the Benjamini-Hochberg method.

### Whole Genome Sequencing (WGS)

WGS data was generated from DNA isolated from whole blood. Illumina Novaseq-6000 machines were used for DNA sequencing to a mean 30× coverage. Raw sequencing reads from FASTQ files were aligned using Burrows-Wheeler Aligner (BWA) (Li and Durbin, 2009) and GVCF files were generated using Sentieon version 201808.03 (Kendig et al., 2019). Functional annotation of variants was done using Variant Effect Predictor from Ensembl (version 101). GATK version 4 (Van der Auwera et al., 2013; DePristo et al., 2011) was used for joint genotyping process and variant quality score recalibration (VQSR). One duplicate sample was removed based on kinship (king cutoff of 0.3) and retained 24,476,739 SNPs that were given a ‘PASS’ filter status by VQSR. For the 72 samples from Critical and Non-Critical groups, there were 15,870,076 variants with MAF<5%. The first ten principal components were generated using plink2 on LD-pruned variants with Hardy-Weinberg equilibrium in controls with a p-value≥1× [symbol] 10 [symbol] \hat{ }(−6) with MAF>5% and were used as covariates to correct for population stratification.

Expression Quantitative Trait Loci (eQTL) Analysis

Local (cis-) expression quantitative trait loci (eQTL) analysis was performed to test for association between genetic variants with gene expression levels for 67 samples having both RNA-seq and SNP genotype data. Briefly, the MatrixEQTL R package (Shabalin, 2012) was used; a linear model was selected and a maximum distance for gene-SNP pairs of 1× [symbol] 10 [symbol] \hat{ }6. The top two principal components identified from the genotype principal component analysis were used as covariates to control for population stratification. 304,044 significant eQTLs were chosen with FDR<=0.05.

### RNA Sequencing (RNA-Seq)

### RNA Extraction

Whole blood RNA was extracted from PAXgene tubes with the PAXgene Blood RNA Kit following the manufacturer's instructions (Qiagen, Germany). A total of 91 samples including 46 Critical, 23 Non-Critical and 22 healthy controls were processed. RNA quantity and quality were assessed using The Agilent 2200 TapeStation system for RIN and Ribogreen for concentration. RNA sequencing libraries were generated using TruSeq Stranded Total RNA with Ribo-Zero Globin kit (Illumina, USA) and sequenced on the Illumina NovaSeq 6000 instrument with S2 flow cells and 151 bp paired-end reads. Raw sequencing data was aligned to a reference human genome build 38 (GRCh38) using short reads aligner STAR (Dobin et al., 2013). Quantification of gene expression was performed using RSEM (Li and Dewey, 2011) with GENCODE annotation v25 (http://www.gencodegenes.org). Raw and processed datasets have been deposited in GEO with identifier GSE172114.

### Differential Gene Expression (DGE) Analysis

For the Critical vs. Non-Critical comparison, DGE analysis was performed for each cut of the train data using a frozen normalization approach to normalize library sizes using the trimmed mean of M-values method (TMM) from the edgeR R package (Robinson and Oshlack, 2010; Robinson et al., 2010). Briefly, low expressed genes were removed for the 69 samples with genes with 1 count per million in less than 10% of samples. For each cut of the train data, the normalization factors were calculated, then the library that had a normalization factor closest to 1 was selected. This was used as a reference library to normalize all samples keeping the training normalization factors unchanged. Differentially expressed genes were identified using a quasi-likelihood F-test (QLF) adjusted P values from edgeR R package. Differentially expressed genes with false discovery rate (FDR) less than 0.05 were used for further downstream analysis.

### Identification of Potential Driver Genes Through Structural Causal Modeling

In order to identify potential bio-markers that may differentiate patients in the Non-critical group from the Critical group, classification as a feature selection approach was used, and then the most informative features were used as input to structural causal modeling to identify potential driver genes. More specifically, classification was performed on the RNA-seq data by repeatedly splitting Non-critical and Critical into 100 unique training and independent test sets representing 80% and 20% of total data, respectively, ensuring that the proportions of Non-critical and Critical patients was consistent in each split of the data. 100 splits of the data were used in order to capture biological variation and have more statistical confidence in the results. After classification, feature scores for each method were determined and combined across all 100 splits of the data and 6 of the machine learning algorithms, not including the deep learning. The top 600 most informative features were retained for structural causal modeling.

The output of the structural causal modeling returned a putative directed network depicting the flow of causal information. In order to incorporate information from other data sources, differential expression for the plasma and PBMC proteomics data was also performed, SKAT for the WGS data, and eQTL and pQTL analysis for the genomic and proteomics data, respectively.

### Ensemble Computational Intelligence

Seven machine learning approaches were used for classification models. The relevant hyper-parameters for each method are mentioned in their respective sections. Hyper-parameters were chosen by using 10-fold cross-validation on the training data, with performance evaluated on the held-out test data.

### Least Absolute Shrinkage and Selection Operator (LASSO), and Ridge Regression

LASSO (Tibshirani, 1996) is an L1-penalized linear regression model defined as:

$$\hat{β} ( λ ) = {min}_{β} [ - log [ L ( y ; β ) ] + λ {\vert \vert β \vert \vert}_{1} \qquad ( 1 )$$

Ridge (Hoerl and Kennard, 1970; Hoerl et al., 1975) is an L₂-penalized linear regression model defined as:

$$\hat{β} ( λ ) = {min}_{β} [ - log [ L ( y ; β ) ] + λ {\vert \vert β \vert \vert}_{2}^{2} \qquad ( 2 )$$

$$where$$

$$L = \frac{1}{N} {\sum }_{i = 1}^{N} {( {y}_{i} - {β}_{0} - {x}_{i} \cdot β )}^{2}$$

In both cases λ>0 is the regularization parameter that controls model complexity. s are the regression coefficients, β₀ is the intercept term, y are the class labels, xᵢ is the ith training sample, and the goal of the training procedure is to determine \hat{β}, the optimal regression coefficients that minimize the quantities defined in Eqs. (1) and (2).

The predicted label is given by ŷ=β₀+x·β, with some threshold introduced to binarize the label for classification problems. In LASSO, the constraint placed on the norm of β (the strength of which is given by λ) causes coefficients of uninformative features to shrink to zero. This leads to a simpler model that contains only a few non-zero coefficients. The ‘glmnet’ function from the caret (Kuhn, 2008) R package was used to train all LASSO and Ridge models.

Ridge plays a similar role in determining model complexity, except that coefficients for uninformative features do not necessarily shrink to zero.

For both LASSO and Ridge, the function over a custom tuning grid of λ from 2⁻⁸ to 2² was implemented. λ was chosen via 10-fold cross-validation as the value that gave the minimum mean cross-validated error.

### Support Vector Machines (SVM)

Support vector machines (SVMs) (Boser et al., 1992; Cortes and Vapnik, 1995) are a set of supervised learning models used for classification and regression analysis. The primal form of the optimization problem is:

$${min}_{w , b , a} {L}_{p} = \frac{1}{2} {\vert \vert w \vert \vert}_{2}^{2} - {\sum }_{i = 1}^{N} {a}_{i} {y}_{i} ( {x}_{i} \cdot w + b ) + {\sum }_{i = 1}^{N} {a}_{i} \qquad ( 3 )$$

where L_{P} is the loss function in its primal form (p for primal), w are the weights to be determined in the optimization, xᵢ is the ith training sample, yᵢ is the label of the ith training sample, aᵢ≥0 are Lagrange multipliers, N is the number of training points, and b is the intercept term. Labels are predicted by thresholding xᵢ·w+b.

The optimization problem in its dual form is defined as:

$${max}_{a} {L}_{D} ( a ) = {\sum }_{i = 1}^{N} {a}_{i} - \frac{1}{2} {\sum }_{i , j = 1}^{N} {a}_{i} {a}_{j} {y}_{i} {y}_{j} K ( {x}_{i} , {x}_{j} ) \qquad ( 4 )$$

where L_{D} is the Lagrangian dual of the primal problem, aᵢ are the Lagrange multipliers, yᵢ and xᵢ are the ith label and training sample, respectively, K(·,·) is the kernel function. Maximization takes place subject to the constraints Σᵢaᵢ yᵢ=0 and aᵢ≥C≥0, ∀i. Here C is a hyper-parameter that controls the degree of misclassification of the model for nonlinear classifiers. The optimal value of w and b can found in terms of the aᵢ's, and the label of a new data point x can be found by thresholding the output Eᵢaᵢ yᵢK(xᵢ, x)+b.

In most cases, many of the a_i's are zero and evaluating predictions can be faster using the dual form. The support vector machines were used with linear kernel (‘svmLinear2’) (i.e., K(x_i,x_j)=x_i·x_j, the inner product of x_i and x_j) function from the caret (Kuhn, 2008) R package to train all SVM models. C ranged from 2\hat{ }(−2) to 2\hat{ }3, and a 10-fold cross-validation was used to tune and select the hyperparameters with the best cross-validation accuracy for training the model.

### Random Forest (RF)

Random Forest (Breiman, 2001; Breiman et al., 1993) is an ensemble learning method for classification and regression which builds a set (or forest) of decision trees. In random forest, n samples are chosen (typically two-thirds of all the training data) with replacement from the training data m times, giving m different decision trees. Each tree is grown by considering ‘mtry’ of the total features, and the tree is split depending on which features gives the smallest Gini impurity. In the event of multiple training samples in a terminal node of a particular tree, the predicted label is given by the mode of all the training samples in a terminal node. The final prediction for a new sample x is determined by taking the majority vote over all the trees in the forest. The ‘rf’ function was used from the caret (Kuhn, 2008) R package to train all Random Forest models. A 10-fold cross-validation was used to tune parameters for training the model. A tune grid with 44 values from 1 to 44 for ‘mtry’, the number of random variables considered for a split each iteration during the construction of each tree, was used for the tuning model.

### XGBoost (XGB)

XGBoost (Chen and Guestrin, 2016) is a distributed gradient boosting library for classification and regression by building an ensemble of decision trees. In contrast to Random Forest, XGBoost uses an additive strategy to add new trees one at a time based on whether they optimize the objective function. The objective function for the t-th tree is:

$${obj}^{( t )} = {\sum }_{j = 1}^{T} [ {G}_{j} {w}_{j} + \frac{1}{2} ( {H}_{j} + λ ) {w}_{j}^{2} ] + γ T$$

where Gⱼ=2 Σ_{i∈I}_{ⱼ}(ŷᵢ^{(t-1)}−yᵢ), Hⱼ=2|Iⱼ|, λ and γ are hyper-parameters controlling model complexity, T is the number of leaves in the trees, wⱼ is the combined score across all the data points for the j-th leaf. Here, Iⱼ refers to the set of indices of data points assigned to the j-th leaf, |Iⱼ| is the size of the set Iⱼ, ŷᵢ^{(t-1)} is the predicted score (without the t-th tree) of the i-th data point, and yᵢ is the actual label of the i-th data point. The default parameter tuning grid in R was used, and a 10-fold cross-validation was used to tune and select the hyperparameters with the best cross-validation accuracy for training the model.
Quantum Support Vector Machines (qSVM)

Quantum support vector machine (qSVM) is a quantum adaptation of SVM that can be used for classification designed to be run with a quantum annealer (QA) (Willsch et al., 2020). The advantage of running the optimization problem on a QA is that, since the QA samples from the quantum distribution, it retains both the lowest energy solution and some of the next lowest-energy solutions. Because of the suboptimal solutions, qSVM is expected to perform worse on the train data than classical SVM (which only includes optimal solution). However, sub-optimal solutions can capture different aspects of train data, and generate different decision boundaries. As such, a suitable combination of the suboptimal solutions in qSVM might outperform cSVM on the test data.

The objective function is the same as for classical SVM up to a change in sign, i.e.,

$${min}_{a} {L}_{D} ( a ) = \frac{1}{2} {\sum }_{i , j = 1}^{N} {a}_{i} {a}_{j} {y}_{i} {y}_{j} K ( {x}_{i} , {x}_{j} ) - {\sum }_{i = 1}^{N} {a}_{i}$$

subject to constraints Σᵢaᵢ yᵢ=0 and aᵢ≥C≥0, ∀i.

qSVM was run on physical quantum annealers manufactured by D-Wave (Johnson et al., 2011). The D-Wave Advantage was used in this work and had 5436 qubits with 15 couplers per qubit, using the Pegasus topology. Since D-Wave can only produce binary solutions, the encoding defined in (Willsch et al., 2020) was used to convert the continuous variables an into K binary variables using base B:

$${α}_{i} = {\sum }_{k = 0}^{K - 1} {B}^{k} {a}_{K i + k} , \qquad {a}_{K i + k} ∈ \{ 0 , 1 \} .$$

Using this encoding and also adding a penalty ξ to the loss function, the optimization problem gets the form of a Quadratic Unconstrained Binary Optimization (QUBO) problem, which can be run on a QA:

$$E = \frac{1}{2} {\sum }_{i , j , k , l} {a}_{K i + k} {a}_{K j + l} {B}^{k + l} {y}_{i} {y}_{j} K ( {x}_{i} , {x}_{j} ) - {\sum }_{i , k} {B}^{k} {a}_{K i + k} + {ξ ( {\sum }_{i , k} {B}^{k} {a}_{K i + k} {y}_{i} )}^{2}$$

$$= {\sum }_{i , j = 0}^{N - 1} {\sum }_{k , l = 0}^{K - 1} {Q}_{K i + l , K j + l} {a}_{K i + k} {a}_{K j + l} ,$$

Where

$${Q}_{K i + k , K j + l} = \frac{1}{2} {B}^{k + l} {y}_{i} {y}_{j} ( K ( {x}_{i} , {x}_{j} ) + ξ ) - {δ}_{i , j} {δ}_{k , l} {B}^{k} .$$

As the objective function above may necessitate connections between any pair of qubits, an embedding is necessary (Choi, 2008). Hyper-parameters were selected using a custom 3-fold Monte-Carlo cross-validation on the train data. Hyper-parameters included the type of kernel (linear versus Gaussian), B (between 2 and 10), K (between 2 and 6), ξ (between 0 and 5), and γ (between 2⁻³ to 2³).

### DANN

Deep learning methodologies were adapted to analyze genomic datasets (Alipanahi et al., 2015) Typical deep neural networks use a series of nonlinear transformations (termed layers), with the final output considered a prediction of class or regression variable. Each layer consists of a set of weights (W) and biases (b) that are tuned during a training phase to learn which nonlinear combinations of input features are most important for the prediction task. These types of models “automatically” learn patterns in the data and combine them, in some abstract nonlinear fashion, to gain an ability to make predictions about the dataset.

The basic formulation of a fully connected DANN is given as

$$For m layers … \{ \begin{matrix}{f}_{1} = {ρ}_{1} ( {\sum }_{j = 1}^{{d}_{1}} ( {W}_{1 , j} \times {X}_{j} ) + {b}_{{d}_{1} + 1} ) \\ {f}_{2} = {ρ}_{2} ( {\sum }_{j = 1}^{{d}_{2}} ( {W}_{2 , j} \times {f}_{1} ) + {b}_{{d}_{2} + 1} ) \\ {f}_{m} = {ρ}_{m} ( {\sum }_{j = 1}^{{d}_{m}} ( {W}_{m , j} \times {f}_{m - 1} ) + {b}_{{d}_{m} + 1} )\end{matrix}$$

where the dimensions of W and b are determined by the number of neurons in each layer (d₁, d₂, . . . , dₘ). Each layer used rectified linear units as activation functions:

ρₗ(z)=max(o,z).

The final layer used a softmax function, with the number of neurons equal to the number of class (K), to convert the logits to probabilities:

$${Φ ( {f}_{m} )}_{j} = \frac{{e}^{{f}_{m , j}}}{{\sum }_{k = 1}^{K} {e}^{{f}_{m , k}}} for j = 1 , … , K ,$$

where f_{m,j} is the output of the j-th neuron of the m-th layer. In addition, the concept of “dropout” was used, which randomly sets a portion of input values (η) to the layer to zero during the training phase (Srivastava et al., 2014). This has a strong regularization effect (essentially by injecting random noise) that helps prevent models from overfitting. Layers that included dropout were formulated as

$$f = p ( {\sum }_{j = 1}^{d} ( {W}_{j} \times {X}_{j} ) + {b}_{d + 1} ) \times {m}_{l} ,$$

where mₗ˜Bernoulli(η).

When evaluating models on test datasets, the dropout mask is not used. The categorical cross-entropy loss function was used to train DANNs, where (Bₙ) is the minibatch size, tᵢ is the correct class index, and pᵢ is the class probability from the softmax layer:

$${L}_{T} = - {\sum }_{i = 1}^{{B}_{n}} {t}_{i} log ( {p}_{i} ) .$$

Minibatch stochastic gradient descent was used with Nesterov momentum to update the DANN parameters based on the loss function above (Sutskever et al., 2013). The TensorFlow (Abadi et al., 2016) python package was used to construct the DANNs.

### Ensemble Feature Ranking

In order to derive an ensemble ranking of the feature importance, feature importances for each algorithm were first calculated. LASSO, Ridge, SVM, and qSVM are linear models, and thus the feature importance was determined based on the value of the weight assigned to each feature, with a larger score corresponding to greater importance. Random Forest creates a forest of decision trees, and as part of the fitting process determines an estimate of the feature importance by randomly permuting the features one at a time and determining the change in the accuracy. XGBoost calculates feature importance by averaging the gain across all the trees, where the gain is the difference in the Gini purity of the parent node and the two children nodes.

The top 1000 most informative features for each model, for each cut of the data were retained for each of the 100 cuts of the training data. Because there were 100 cuts of the data, 6 algorithms (LASSO, Ridge, SVM, qSVM, RF, and XGBoost; DANN was not included because it lacks a robust approach to determine feature importance), and up to 1000 features retained, a total of up to 600,000 possible features were considered for each feature set (though they may not be unique, as the top 1000 features for one cut of the data may have some overlap with the top 1000 features for another cut of the data). Feature scores from an algorithm on any cut that had a test AUROC<0.7 were discarded, in an attempt to exclude scores that may not truly be informative. To aggregate the scores, the scores were scaled by the most informative feature for each algorithm on each cut, such that the feature scores all lay between 0 and 1, i.e., for the first cut of the data the 1000 most informative features from LASSO were scaled, then the same was done for Ridge, SVM, Random Forest, and the process repeated for each cut of the data. Scores were then averaged across all the cuts of the data to give a feature ranking for each method. If a feature was determined to be important for one cut of the data but not for others, it was given a value of 0 for all cuts of the data in which it did not appear. To determine a final ensemble feature ranking, the grand mean across all training cuts and algorithms was taken, and the features were sorted by the average score.

### Structural Causal Modeling

BBNs were generated for the top 600 most informative genes as defined by ensemble feature ranking described above. BBNs were used to assess the conditional dependence and probabilistic relationships between the most informative genes. Briefly, a minibatch stochastic gradient descent with Nesterov momentum was used to update the DANN parameters based on the loss function above (Sutskever et al., 2013). The TensorFlow (Abadi et al., 2016) python package was used to construct the DANNs. G. A set of common assumptions to determine the causal structure were relied upon: (1) causal sufficiency assumption, where there are no unobserved cofounders; (2) causal Markov assumption, where all d-separations in the graph (G) imply conditional independence in the observed probability distribution; and (3) causal faithfulness assumption, where all of the conditional independences in the observed probability distribution imply d-separations in the graph (G). Notably, the data may not strictly meet all of these assumptions, however the generated BBNs provide useful biological hypothesis that could be experimentally validated.

BBNs were determined using the bnlearn R package with the score-based hill-climbing algorithm that heuristically searched the optimality space of all possible DAGs (Scutari, 2010). As the hill-climbing algorithm can get trapped in local optima and is quite dependent on the starting structure, 100 BBNs starting from different network seeds were initialized. During the hill-climbing process, each candidate BBN was assessed with the Bayesian information criterion (BIC) score (Lam and Bacchus, 1994; Scutari, 2010):

$$BIC = log L ( {X}_{1} , … , {X}_{v} ) - \frac{d}{2} log n ,$$

where X₁, . . . , Xᵥ is the node set, d is the number of free parameters, n is the sample size of the dataset, and L is the likelihood. This definition of the BIC, which is the version implemented in the bnlearn package, rescales the classic definition by −2. The penalty term was used to prevent overly complicated structures and overfitting. Each run of the hill-climbing algorithm returns a structure that maximizes the BIC score (including evaluating the directions of edges). A caveat is that these structures may be partially oriented graphs (i.e., situations where the directionality of some edges cannot be effectively determined). The cextend function from the bnlearn package was used to construct a DAG that is a consistent extension of X. A consensus network based on the 100 networks after hill-climbing was then generated, wherein edges that were present in graphs at least 30% of the time were kept. Any residual undirected edges contained in the consensus network were discarded. Statistical significance of edges within the imposed consensus network was assessed by randomly permuting the dataset 10,000 times and evaluating the consensus structure on these scrambled datasets (thus providing an estimate of the null distribution). BBN edges with a false discovery rate of 5% (i.e., the edge occurred in ≥500 of the random BBNs) or greater were removed from the final network.

After deriving a final consensus network structure, a series of in silico tests to determine the importance of each gene to the network was performed. For each of the 600 genes, all incident edges were removed (both incoming and outgoing) and the BIC of the entire network was recalculated. Doing so resulted in a lower BIC, and the magnitude of the change in BIC is a measure of how important a gene is to the network. Experimentation with permuting the data corresponding to a single gene was performed and the results for the mean change in BIC using the permutation test and removing all the incident edges did not significantly differ (Pearson's correlation >0.999). Having derived a measure for the importance of each gene to the network, the mean change in BIC of the top 5 driver genes can be compared to 1000 random sets of 5 genes from the network.

### Real-Time Reverse Transcription Quantitative PCR (RT-qPCR)

Total RNA was extracted from cells with the RNeasy Mini Kit (Qiagen, Germany), and RNA quality was assessed using an Agilent2100 BioAnalyzer before reverse transcription into cDNA with Maxima™ H Minus Mastermix and following the manufacturer's instructions (ThermoFisher Scientific, USA). RT-qPCR was performed using QuantStudio3 (ThermoFisher Scientific, USA) according to the manufacturer's protocol, and using PowerTrack™ SYBR™ Green Master Mix (ThermoFisher Scientific, USA). The following primers were used for ADAM9: forward 5′-GGACTCAGAGGATTGCTGCATTTAG-3′ (SEQ ID NO: 1), reverse 5′-CTTCGAAGTAGCTGAGTCATGCTGG-3′ (SEQ ID NO: 2) and GAPDH as a housekeeping gene: forward

5′-GGTGAAGGTCGGAGTCAACGGA-3′ (SEQ ID NO: 3) and 5′-GAGGGATCTCGCTCCTGGAAGA-3′ (SEQ ID NO: 4) (Integrated DNA Technologies, USA). The RT-qPCR protocol consisted of: 95° C. for 2 min, followed by 40 cycles: 95° C. for 5 sec and 60° C. for 30 sec. All reactions were performed in duplicate and the relative amounts of transcripts were calculated with the comparative Ct method. Gene expression changes were calculated using 2^{·ΔΔCt} values calculated from averages of technical duplicates, relative to the negative control. Melting-curve analysis was performed to assess the specificity of the PCR products.

### Enzyme-Linked Immunosorbent Assays (ELISA)

Soluble ADAM9 (sADAM9) and soluble MICA (sMICA) were quantified by ELISA on serum of Critical patients, Non-Critical patients and healthy controls. For soluble ADAM9, Human sADAM9 DuoSet ELISA kit (R&D Systems, Minneapolis, MN, USA) was used following manufacturer's instructions. sMICA levels were measured with an in-house developed sandwich enzyme-linked immunosorbent assay (ELISA) using two monoclonal mouse antibodies for capture (A13-C485B10 and A9-C255A9 at 2 mg/ml and 0.2 mg/ml, respectively) and one biotinylated monoclonal mouse antibody for detection (A15-C199B9 at 60 μg/ml). Coating of MaxiSorp ELISA plates (ThermoFisher Scientific, Waltham, MA, USA) was performed in PBS at 4° C. overnight. After three washing steps with PBS, the wells were blocked with 200 ml of 10% BSA in PBS for 1 h at room temperature. All the following steps were carried out at room temperature with PBS/0.05% Tween 20/10% BSA used as a diluent for all the reagents and sera. The plates were washed three times with PBS/0.05% Tween 20 between incubation steps. After blocking, the plates were incubated with 100 ml of sera, standards and controls for 2 h, followed by incubation with 100 ml biotinylated detection antibody for 1 h. Then the plates were incubated during 1 h with 100 ml of a 5000-fold dilution of streptavidin poly-HRP (ThermoFisher Scientific, USA) per well. The reactions were finally revealed using TMB Ultra (ThermoFisher Scientific, USA) at 100 ml/well for 15 min and stopped with 100 ml of 1M HCl. The absorbance was measured at 450 nm.

### Cell Culture

Vero E6 cell lines were grown at 37° C. under 5% CO₂ and maintained in DMEM Medium (ThermoFisher Scientific, USA) containing 100 units/ml penicillin, which was supplemented with 10% fetal bovine serum (Pan Biotech, Germany). ACE2-expressing A549 cells (A549-ACE2) were grown at 37° C. under 5% CO₂ and maintained in DMEM Medium (ThermoFisher Scientific, USA) containing 10 μg/ml of Blasticidine S (Invitrogen, USA).

### Silencing and Cell Transfection

Cells were transfected with predesigned Stealth siRNA directed against ADAM9 (HSS112867) or the control Stealth RNAi Negative Control Duplex medium GC (45-55%) (ThermoFisher Scientific, USA) by using Lipofectamine™ 3000 Reagent (ThermoFisher Scientific, USA). One day prior to transfection, the cells were seeded in a 24-well plate at 0.05×10⁶ cells per well. First 1.5 μl of Lipofectamine™ 3000 Reagent were added to 25 μl of Opti-MEM™ medium, followed by addition of the mix containing 5 pmoles of siRNA in 25 μl of Opti-MEM™ medium (ThermoFisher Scientific, USA). The mixture was incubated at room temperature for 10 min and then added to the cells. The cells were collected or infected after 48 h.

### Western Blot

After collection and centrifugation, cells were washed once in Dulbecco's Phosphate Buffered Saline (D-PBS, Sigma Aldrich, USA). The pellet was resuspended in 60 μl of RIPA lysis buffer (150 mM NaCl, 5 mM EDTA, 1% NP40, 50 mM Tris pH 8, 0.5% sodium deoxycholate, 0.1% SDS) including protease inhibitors (cOmplete, Roche Diagnostics, Switzerland) and left on ice during 20 min. The total cellular extract was then centrifuged during 30 min at 13,000 g to remove all cell debris. A Bradford assay was performed for quantifying proteins (BIO-RAD protein Assay, Bio-Rad Laboratories, USA). For western blotting analysis, 20 μg of total cell extract was loaded on a 8% SDS-poly-acrylamide gel. After migration, proteins were transferred onto a PVDF membrane with a semi-dry transfer system (Trans-Blot, Bio-Rad Laboratories, USA). Membranes were blocked during 1 h in 5% skimmed milk/PBS 0.05%/tween20 and then incubated with the anti-ADAM9 antibody (ab218242; Abcam, UK) during 2 h at 4° C. in 5% BSA/TBS 0.1% tween at 1/1000 dilution. The membrane was then incubated with the secondary antibody coupled to HRP Bio-Rad Laboratories, USA). Bound antibodies were revealed with an enhanced chemiluminescence detection system using the ChemiDoc XRS (Bio-Rad Laboratories, USA). Loading control was performed with an anti-GAPDH antibody (MAB374, Merck Millipore, USA).

### In Vitro Viral Infections

Vero E6 and A549-ACE2 cell lines were infected with SARS-CoV-2 wild type virus at MOI of 10 and 400, respectively. Percentage of infected cells was determined by staining with SARS-CoV-2 Nucleocapsid (% of Nucleocapsid positive cells) and virus released in the supernatant was analyzed by RT-PCR (copies/ml) after 2 and 3 days of infection for Vero E6 and A549-ACE2 cells, respectively.

### Flow Cytometry Stainings

Cells were fixed with for 20 min in 3.6% paraformaldehyde at 4° C., washed in PBS 5% Fetal Calf Serum (FCS) and stained with anti-nucleocapsid Antibody (GTX135357, Genetex, USA) at 1/200 dilution in permwash (Becton, Dickinson and Company, USA) for 45 min at room temperature. The antibody was then revealed by incubation with a Alexa 647-labeled goat anti-Rabbit monoclonal antibody (Ab150083, Abcam, UK) diluted at 1/200 in PBS 5% FCS for 45 min at room temperature.

### Viral Real-Time Reverse Transcription Quantitative PCR (RT-qPCR)

RNA was extracted from the supernatant of infected cells with the NucleoSpin Dx Virus Kit (Macherey-Nagel GmbH & Co.KG, Germany). RT-qPCR was performed using TaqPath™ 1-Step RT-qPCR Master Mix, CG on the Quanstudio3 instrument (ThermoFisher Scientific, USA). The primer/probe mix used for absolute quantification of the virus are N1 and N2 from the 2019-nCoV RUO Kit (Integrated DNA Technologies, USA), and the positive control for the standard curve was 2019-nCoV N Positive Control (Integrated DNA Technologies, USA). The reaction was performed in 20 μl, including 5 μl of eluted RNA, 5 μl of TaqPath master mix and 1.5 μl of primer/probe. The RT-qPCR protocol consisted of: 25° C. for 2 min, 50° C. for 15 min, 95° C. for 2 min, followed by 40 cycles: 95° C. for 3 sec and 60° C. for 30 sec. All reactions were performed in duplicate and the absolute quantification was calculated with the standard curve of the positive control.

### Example 2: Patient Characteristics and Study Design

Study participants were selected from patients that were hospitalized for COVID-19 in a university hospital network in northeast France (Alsace) during the first European wave of the pandemic (March-April 2020), before routine use of corticosteroids. A total of 72 patients under 50 years of age and without major comorbidities were enrolled. Among these, 53 were men (73.6%) with a median age of 40 [IQR 33; 46] years. The patients were divided into two groups:
(i) a “critical” group consisting of 47 patients who were hospitalized in the ICU due to ARDS (44 patients, 60.3%) or severe symptomatology (3 patients, 4.1%) needing invasive mechanical ventilation, and
(ii) a “non-critical” group consisting of 25 patients (34.2%) who stayed in a non-critical care ward. In the latter group, 19 (76%) received oxygen support.
   Patients who were transferred from the non-critical care ward to the ICU were considered as critical. For ICU patients, the median of simplified acute physiology score (SAPS) II was 38 [IQR 33; 47] points and median PaO₂/FiO₂ was 123 [IQR 95; 168] mmHg on admission. All patients were discharged from the hospital or were deceased at the time of data analysis. The hospital day-28 mortality rate in the critical group was 13% (6 patients). Patient characteristics of both groups are summarized in Table 1 and specific ICU patients' characteristics are summarized in Table 2.

TABLE 1
Characteristics of patients admitted in hospital for COVID-19

| | All patients | | Non-critical | | Critical Group | | |
|---|---|---|---|---|---|---|---|
| | (n = 72) | | Group (n = 25) | | (n = 47) | | P |
| Age - median, IQR | 40 | [33; 46] | 38 | [31; 45] | 41 | [34; 46] | 0.24 |
| Male - n (%) | 53 | (73.6) | 17 | (68.0) | 36 | (76.6) | 0.61 |
| BMI (kg/m²) - median, IQR | 30.0 | [26.8; 35.0] | 29.7 | [23.8; 33.0] | 30.2 | [27.1; 35.6] | 0.54 |
| Time since first symptoms (days) - median, | 8.0 | [6.0; 11.0] | 9.5 | [7.2; 13.5] | 7.0 | [6.0; 10.0] | 0.08 |
| IQR | | | | | | | |
| Non-steroidal anti-inflammatory drug <7 | 2 | (2.8) | 1 | (4.0) | 1 | (2.1) | 1.00 |
| days - n (%) | | | | | | | |
| COVID-19 treatments - n (%) | | | | | | | |
| Lopinavir/Ritonavir | 21 | (29.1) | 3 | (12.0) | 18 | (38.3) | 0.02 |
| Remdesivir | 3 | (4.1) | 1 | (4.0) | 2 | (4.2) | 1.00 |
| Hydroxychloroquine | 19 | (26.4) | 2 | (8.0) | 17 | (36.2) | 0.01 |
| Corticosteroids | 6 | (8.3) | 1 | (4.0) | 6 | (12.8) | 0.25 |
| Anti-IL6R or placebo* | 2 | (2.8) | 2 | (8.0) | 0 | | 0.12 |
| Neurological symptoms - n (%) | 26 | (50.0) | 10/25 | (40.0) | 16/27 | (59.2) | 0.27 |
| Outcome - n (%) | | | | | | | |

| | | | | | | |
|---|---|---|---|---|---|---|
| Mortality | 6 | (8.3) | 0 | 6 | (12.8) | 0.09 |

BMI: body mass index; IL-6: interleukin 6, IQR: interquartile range.
*patients included in a randomized control trial.

TABLE 2
Characteristics of ICU patients

| | Critical Group | |
|---|---|---|
| | (n = 47) | |
| Baseline severity scores | | |
| SAPS II - median, IQR | 38 | [33; 47] |
| SOFA - median, IQR | 6 | [4; 9] |
| ARDS - n (%) | 45 | (95.7) |
| Moderate | 21 | (46.7) |
| Severe | 24 | (53.3) |
| Supportive treatments | | |
| Invasive mechanical ventilation - n (%) | 45 | (95.7) |
| Duration of invasive mechanical ventilation | 13 | [7; 24] |
| (days) - median, IQR | | |
| NMBA - n (%) | 40 | (89.0) |
| Catecholamines - n (%) | 41 | (91.1) |
| Catecholamines (days) - median, IQR | 4 | [2; 10] |
| RRT - n (%) | 7 | (15.6) |
| ECMO - n (%) | 2 | (4.4) |

ARDS: acute respiratory distress syndrome, ECMO: extracorporeal membrane oxygenation, IQR: interquartile range, NMBA: neuromuscular blocking agent, RRT: renal replacement therapy, SAPSII: simplified acute physiology score II, SOFA: Sequential Organ Failure Assessment.

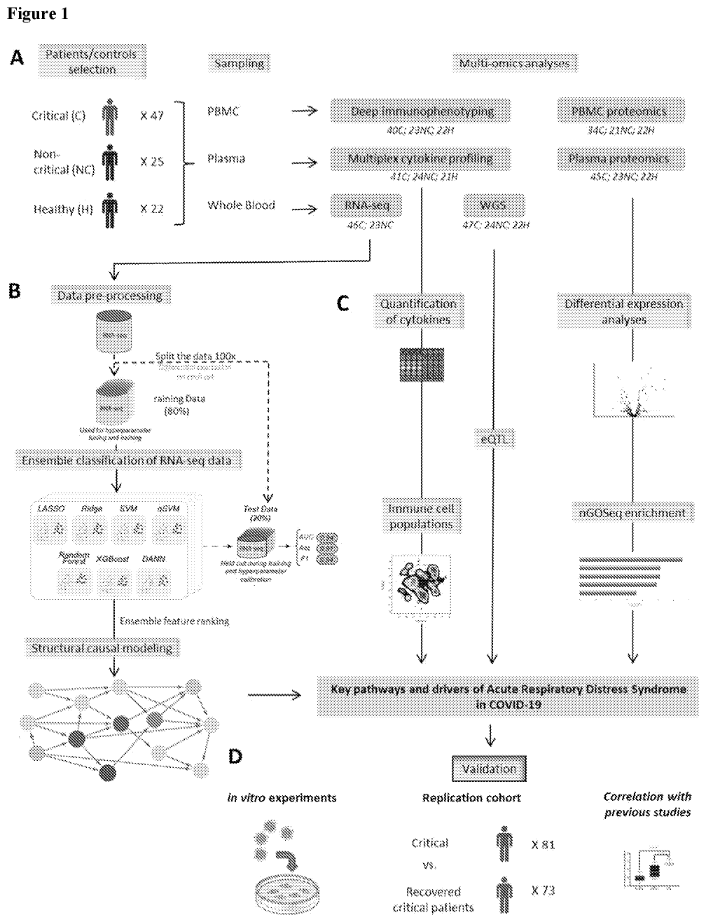

Based on these two patient groups and an additional group of 22 healthy sex- and aged-matched controls, a global multi-omics analysis strategy was used to identify pathways and drivers of ARDS (FIG. 1). Peripheral Blood Mononuclear Cells (PBMC) were analyzed by mass-cytometry (CyTOF®) and whole proteomics. Plasma samples were used for multiplex cytokine quantification and whole proteomics. Finally, RNA-seq and WGS was performed on whole blood. Unless otherwise specified, all measures were made on samples that were taken at the time of entry into the ICU or the non-critical care ward. Validation of the identified driver genes and pathways was performed using an ex vivo model of SARS-CoV-2 infection and a validation cohort of 81 critical patients and 73 recovered critical patients.

### Example 3: Cytokines, Antibodies, and Immune Cell Hallmarks of Critical COVID-19

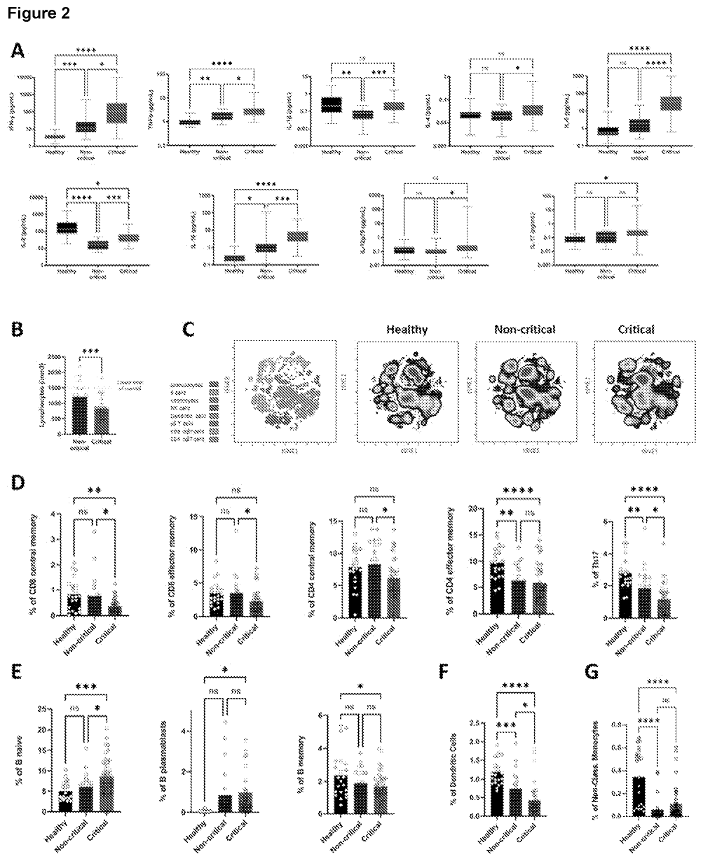

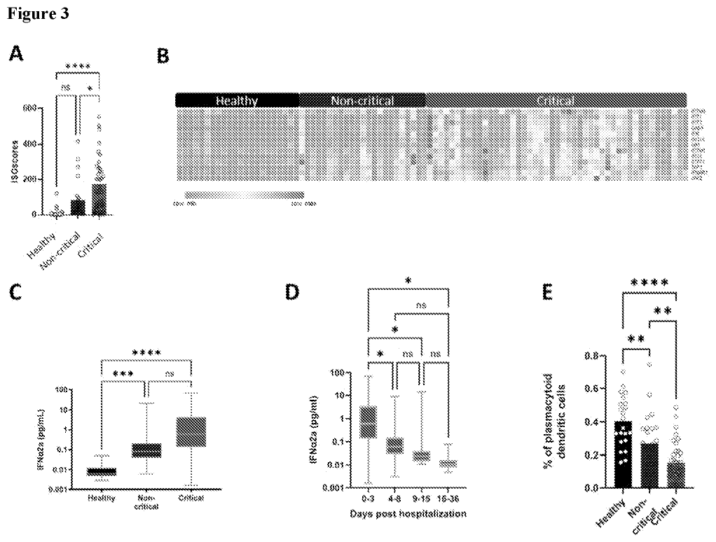

The global pro-inflammatory cytokine profile showed a significantly increased concentration of IFNγ, TNFα, IL-1β, IL-4, IL-6, IL-8, IL-10 and IL-12p70 in critical versus non-critical patients (FIG. 2A). This “cytokine storm” (Mehta et al., 2020) is more pronounced in critical cases, as only IFNγ, TNFα and IL-10 are higher in non-critical patients as compared to healthy controls. Although the disease severity was initially associated with an RNA-seq based type I IFN signature, the absence of a significant increase of the plasma level of IFNα in critical versus non-critical patients, the diminution of the IFNα concentration during the ICU stay and the decreased number of plasmacytoid dendritic cells, the main source of IFNα, suggest that the IFN response is indeed impaired in critical patients (FIG. 3) (Hadjadj et al., 2020; Zhang et al., 2020).

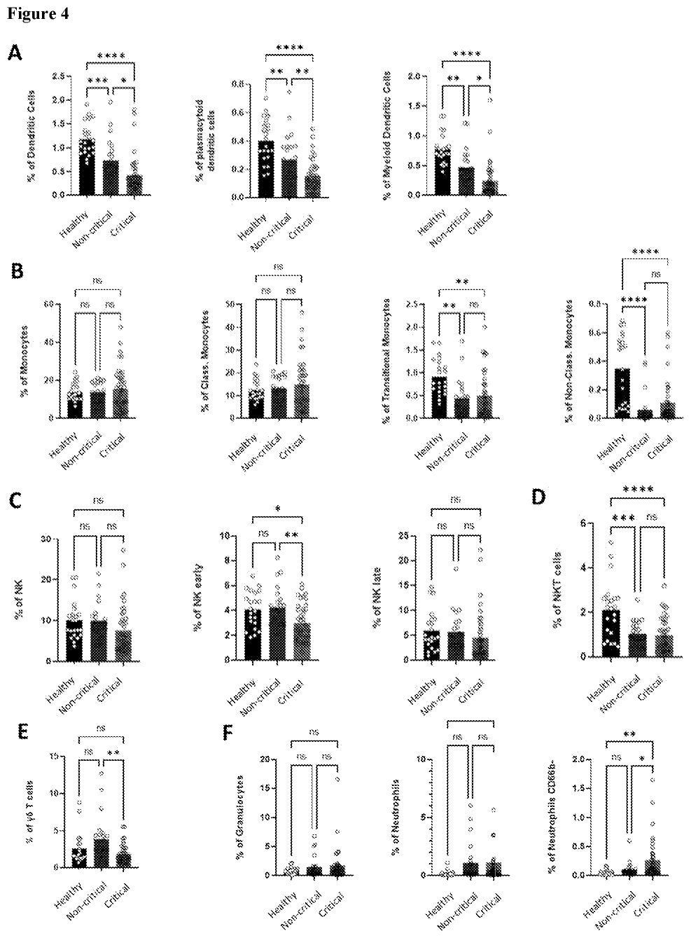

At a systemic level, lymphopenia correlated with disease severity (Guan et al., 2020; Huang et al., 2020; Mehta et al., 2020) (FIG. 2B). To further characterize the immune cells, PBMC were analyzed by mass cytometry using an immune profiling assay covering 37 cell populations. Visualization of stochastic neighbor embedding (viSNE) showed a cell population density distribution pattern that was specific to the critical group (FIG. 2C). This could be partly linked to the known immunosuppression phenomenon in severe patients (Hadjadj et al., 2020; Leisman et al., 2020; Remy et al., 2020), which was characterized by marked differences in the T cell compartments where memory CD4, memory CD8 and Th17 cells negatively correlated with disease severity (FIG. 2D). The latter observation is in line with the absence of a clear association of plasmatic level of IL-17 with severity (FIG. 2A). In the B cell compartments, conversely, there were more naïve B cells and plasmablasts and fewer memory B cells in critical patients versus healthy controls (FIG. 2E). There was a tendency for a higher number of plasmablasts in critical versus non-critical patients. Non-critical and critical patients were also characterized by a lower number of dendritic cells and non-classical monocytes (FIGS. 2F and 2G). The remaining cell populations are presented in the FIG. 4. Altogether, critical illness was characterized by a pro-inflammatory cytokine storm and changes in cell populations that involve mainly T cells, B cells, dendritic cells and monocytes. These specific changes were independent from the extent of viral infection per se, as both the global anti-SARS-CoV-2 antibody levels and their neutralizing activity were not significantly different in critical versus noncritical patients.

### Example 4: Quantitative Plasma and PBMC Proteomics Highlight Signatures of Acute Inflammation, Myeloid Activation and Blood Coagulation

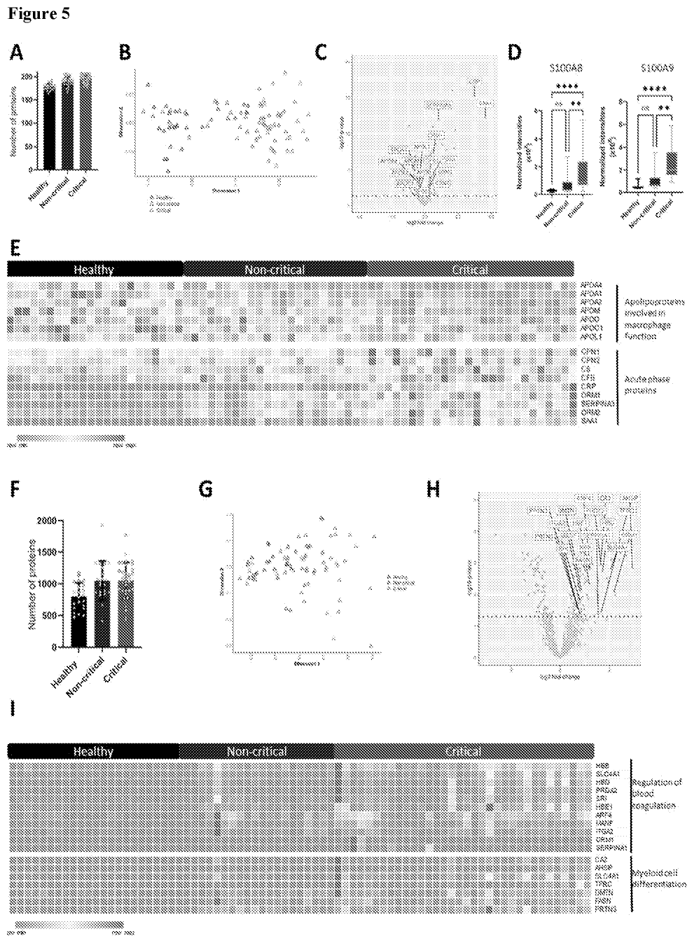

Quantitative nano LC-MS/MS analysis of whole plasma samples identified an average of 178±7, 189±11 and 195±8 proteins in healthy individuals, non-critical and critical patients, respectively (FIG. 5A). After validating the homogeneous distribution of the three groups using a multidimensional scaling plot, differential protein expression analysis was performed in order to identify protein signatures that were specific to critical patients (FIGS. 5B and 5C). In line with previous studies (Chen et al., 2020b; Silvin et al., 2020), the antimicrobial calprotectin (heterodimer of S100A8 and S100A9) was among the top differentially expressed proteins (DEPs) in critical vs. non-critical patients, which confirms that it is a robust marker for disease severity (FIG. 3D). The data also showed a dysregulation of multiple apolipoproteins including APOA1, APOA2, APOA4, APOM, APOD, APOC1 and APOL1 (FIGS. 5C and 5E). Most of them were associated with macrophage functions and were down-regulated in critical patients. Acute phase proteins (CRP, CPN1, CPN2, C6, CFB, ORM1, ORM2, SERPINA3 and SAA1) were strongly up-regulated in critical patients (FIGS. 5C and 5E). These findings are consistent with previous studies reporting that acute inflammation and excessive immune cell infiltration are associated with disease severity (Chen et al., 2020c; Guan et al., 2020; Shu et al., 2020).

Whole cell lysates of PBMC from the same groups of patients and controls were also subjected to quantitative nano LC-MS/MS analysis. An average of 801±213, 1050±309 and 1052±286 proteins were identified and quantified in PBMC of healthy donors, non-critical patients and critical patients, respectively (FIG. 5F). Although the distribution of the three groups in the multidimensional scaling plot is less clear than for plasma proteins, the differential expression analysis between non-critical and critical patients showed a dysregulation of blood coagulation and myeloid cell differentiation (FIGS. 5G, 5H and 5I). The latter observation involving the CA2, AHSP, SLC4A1, TFRC, DMTN, FASN and PRTN3 proteins was in line with the plasma proteomics results evidencing dysregulation of macrophages and with other reports showing that severe COVID-19 is marked by a dysregulated myeloid cell compartment (Schulte-Schrepping et al., 2020). The profile of blood coagulation proteins HBB, HBD, HBE1, SLC4A1, PRDX2, SRI, ARF4, MANF, ITGA2, ORM1 and SERPINA1 confirmed that severity is also associated with coagulation-associated complications that can be either bleeding or thrombosis (Al-Samkari et al., 2020).

### Example 5: Combined Transcriptomics and Proteomics Analysis Supports Inflammatory Pathways Associated with Critical Disease

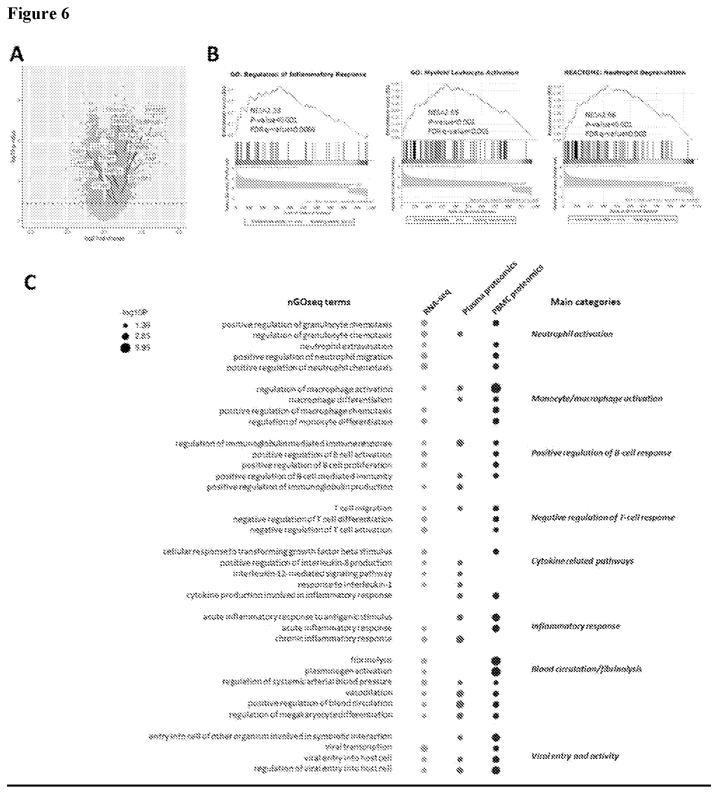

In accordance with proteomics data, differential gene expression and gene set enrichment analysis of RNA-seq data from whole blood of patients showed that regulation of the inflammatory response, myeloid cell activation and neutrophil degranulation are major enriched pathways in critical patients with normalized enrichment scores of 2.33, 2.65 and 2.66, respectively (FIGS. 6A and 6B).

To identify enriched pathways that were supported by different omics-layers, nested GOSeq (nGOseq Nature 2017 May 11; 545(7653):224-228) functional enrichment was performed on differentially expressed genes or proteins in RNA-seq, plasma and PBMC proteomics data. FIG. 6C shows the nGOseq terms that were statistically enriched in at least two omics datasets in critical vs. non-critical patients. In line with cytokine profiling (FIG. 2A), inflammatory signaling and response to pro-inflammatory cytokine release (IL-1, IL-8 and IL-12) were supported by multiple omics datasets. As already suggested by immune cell profiling (FIGS. 2C and 2D) and previous studies, the B-cell response was activated, whereas the T cell response was impaired (De Biasi et al., 2020a; Li et al., 2021). As previously shown (Meizlish et al., 2021; SAnchez-Cerrillo et al., 2020; Schulte-Schrepping et al., 2020; Silvin et al., 2020), activation of neutrophils and monocytes was confirmed by enrichment of nine different nGOseq terms (FIG. 4). The nGOseq enrichment also indicated that the dysfunction in blood coagulation involves a fibrinolytic response, an observation that could, however, be linked to the anti-coagulant therapy of most critical patients (91% of critical patients vs. 56% of non-critical patients were treated with heparin). Finally, nGOseq terms related to viral entry and even viral transcription were strongly enriched in the three omics datasets. This result was concordant with the identification of viral gene transcripts in RNA-seq data of 8 critical patients but not in non-critical patients (Table 3).

TABLE 3
Critical patients in whom viremia could be detected and
their corresponding FPKM values per SARS-CoV-2 gene

| Sample | FPKM* | ORF | ORF | | | ORF | | ORF | ORF | | ORF | ORF | ORF |
|---|---|---|---|---|---|---|---|---|---|---|---|---|---|
| ID | mean | 1ab | 1ab | S | N | 3a | M | 8 | 7a | E | 6 | 7b | 10 |
| P14 | 0.0008333 | 0 | 0.01 | 0 | 0 | 0 | 0 | 0 | 0 | 0 | 0 | 0 | 0 |
| P27 | 0.0008333 | 0 | 0.01 | 0 | 0 | 0 | 0 | 0 | 0 | 0 | 0 | 0 | 0 |
| P31 | 0.0125 | 0 | 0 | 0.01 | 0 | 0 | 0 | 0 | 0.14 | 0 | 0 | 0 | 0 |
| P32 | 0.0025 | 0 | 0 | 0 | 0.03 | 0 | 0 | 0 | 0 | 0 | 0 | 0 | 0 |
| P37 | 0.2683333 | 0.14 | 0 | 0.18 | 0.41 | 0.08 | 0.52 | 0.13 | 0.13 | 0.35 | 1.28 | 0 | 0 |
| P39 | 0.0175 | 0 | 0.01 | 0.03 | 0 | 0 | 0.05 | 0 | 0.12 | 0 | 0 | 0 | 0 |
| P43 | 0.0066667 | 0.01 | 0 | 0 | 0.07 | 0 | 0 | 0 | 0 | 0 | 0 | 0 | 0 |
| P46 | 0.02 | 0.02 | 0 | 0.04 | 0.15 | 0.03 | 0 | 0 | 0 | 0 | 0 | 0 | 0 |

*FPKM: fragments per kilo per million

### Example 6: Integrated Ensemble AI/ML and Probabilistic Programming Discovers a Robust Expression Gene Signature and Driver Genes that Differentiate Critical from Non-Critical Patients

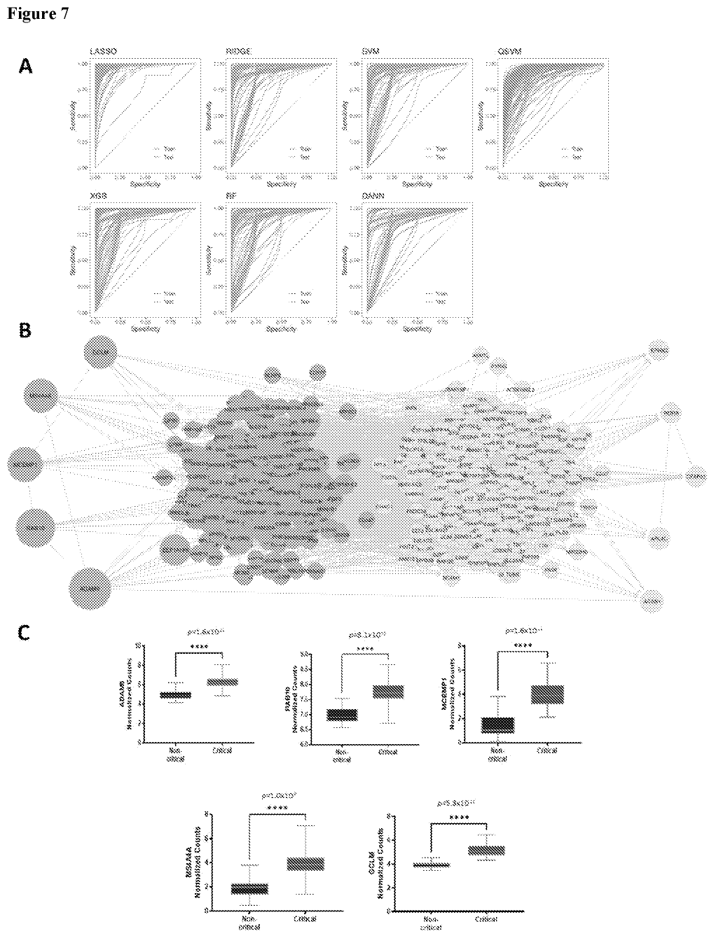

In order to robustly identify a set of genes that may differentiate between non-critical and critical COVID-19 patients and thereby is related to the progression of ARDS, the pipeline depicted in FIG. 1 was adopted. Briefly, patient blood RNA-seq data was partitioned 100 times in order to account for sampling variation, using 80% for training and 20% for testing, and evaluated the performance of seven distinct classes of AI/machine learning (ML) algorithms, including a quantum Support Vector Machine (qSVM) to differentiate between non-critical and critical COVID-19 patients. Quantum annealing is a more robust classifier for relatively small patient training sets (Li et al., Patterns, in press). The Receiver Operating Characteristic curves (ROCs) for the 100 partitions of patient data as well as other classification performance metrics are shown in FIG. 7A and Table 4. The classification performance on the test set provided a high degree of confidence that the signals learned by the various AI/ML algorithms are generalizable.

TABLE 4
Performance metrics on the train and test set for each algorithm
in the ensemble computational intelligence approach.

| | LASSO | Ridge | SVM | qSVM | XGB | RF | DANN |
|---|---|---|---|---|---|---|---|
| Accuracy | 0.9991 ± | 1.0000 ± | 1.0000 ± | 0.9245 ± | 0.9952 ± | 1.0000 ± | 1.0000 ± |
| (Train/Test) | 0.0004/ | 0.0000/ | 0.0000/ | 0.0028/ | 0.0008/ | 0.0000/ | 0.0000/ |
| | 0.9677 ± | 0.9169 ± | 0.9223 ± | 0.8677 ± | 0.9146 ± | 0.9254 ± | 0.9131 ± |
| | 0.0050 | 0.0072 | 0.0075 | 0.0121 | 0.0076 | 0.0072 | 0.0083 |
| Balanced Acc. | 0.9987 ± | 1.0000 ± | 1.0000 ± | 0.9189 ± | 0.9930 ± | 1.0000 ± | 1.0000 ± |
| (Train/Test) | 0.0006/ | 0.0000/ | 0.0000/ | 0.0039/ | 0.0012/ | 0.0000/ | 0.0000/ |
| | 0.9503 ± | 0.8990 ± | 0.9068 ± | 0.8607 ± | 0.8932 ± | 0.9072 ± | 0.9032 ± |
| | 0.0078 | 0.0094 | 0.0092 | 0.0118 | 0.0100 | 0.0094 | 0.0097 |
| AUROC | 1.0000 ± | 1.0000 ± | 1.0000 ± | 0.9667 ± | 0.9999 ± | 1.0000 ± | 1.0000 ± |
| (Train/Test) | 0.0000/ | 0.0000/ | 0.0000/ | 0.0029/ | 0.0000/ | 0.0000/ | 0.0000/ |
| | 0.9908 ± | 0.9547 ± | 0.9633 ± | 0.9386 ± | 0.9443 ± | 0.9360 ± | 0.9435 ± |
| | 0.0036 | 0.0075 | 0.0070 | 0.0081 | 0.0079 | 0.0091 | 0.0081 |
| F1 | 0.9993 ± | 1.0000 ± | 1.0000 ± | 0.9426 ± | 0.9964 ± | 1.0000 ± | 1.0000 ± |
| (Train/Test) | 0.0003/ | 0.0000/ | 0.0000/ | 0.0020/ | 0.0006/ | 0.0000/ | 0.0000/ |
| | 0.9780 ± | 0.9404 ± | 0.9487 ± | 0.9095 ± | 0.9391 ± | 0.9467 ± | 0.9359 ± |
| | 0.0034 | 0.0052 | 0.0049 | 0.0071 | 0.0054 | 0.0052 | 0.0062 |
| MCC | 0.9980 ± | 1.0000 ± | 1.0000 ± | 0.8339 ± | 0.9893 ± | 1.0000 ± | 1.0000 ± |
| (Train/Test) | 0.0009/ | 0.0000/ | 0.0000/ | 0.0065/ | 0.0018/ | 0.0000/ | 0.0000/ |
| | 0.9251 ± | 0.8128 ± | 0.8364 ± | 0.7398 ± | 0.8061 ± | 0.8308 ± | 0.8091 ± |
| | 0.0118 | 0.0169 | 0.0161 | 0.0198 | 0.0181 | 0.0168 | 0.0185 |

After successfully classifying non-critical versus critical patients based on whole-transcriptome RNA-seq profiling, feature scores were assessed across the six distinct ML algorithms (see Methods) and all partitions of patient data to determine an ensemble feature ranking, ignoring features from the partitions of patient data where the test AUROC was less than 0.7. Aggregating the best performing features across both the algorithm and data partitions afforded a more robust and stable set of generalizable features.

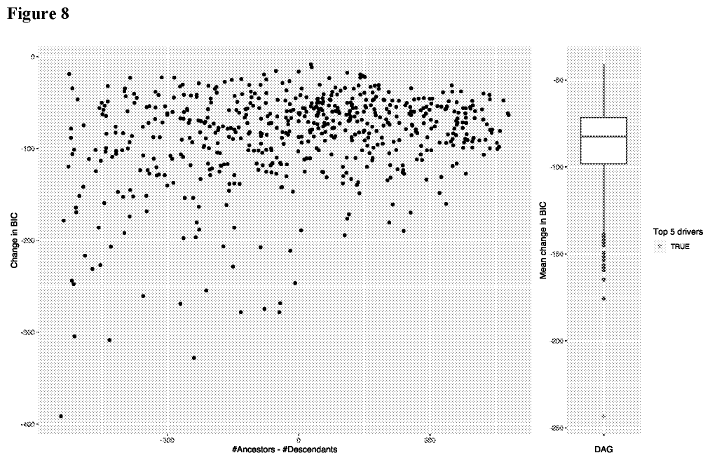

This signature represents hundreds of genes that are differentially expressed and by itself does not distinguish between driver genes of severe COVID-19 and genes that react to the disease. Therefore, the top 600 most informative genes were then selected and used as input for structural causal modeling (SCM) to find likely drivers of severe COVID-19 disease. Previous work has shown that SCM of RNA-seq data produces causal dependency structures, indicative of the signal transduction cascades that occur within cells and drive phenotypic and pathophenotypic development (Ricard et al., J Exp Med, 2019). However, this approach works best if the gene sets are stable and consistent across 7 different algorithms as shown herein. The resultant SCM output is presented as a directed acyclic graph (DAG) in FIG. 7B, a gene network representing the putative flow of causal information, with genes on the left predicted to have the greatest degree of influence on the entire state of the network. Perturbing these genes is most disruptive to the state of the network (FIG. 8), and is expected to have the greatest effect on the expression of downstream genes. The top five genes that associated with the greatest degree of putative causal dependency are ADAM9, RAB10, MCEMP1, MS4A4A and GCLM, all five being significantly up-regulated in critical patients (FIG. 7C). The DAG also shows 5 downstream genes at the right of the graph in FIG. 7B (EPHX2, RORA, CFAP97, ARL4C or ACSS1) which are predicted to have the greatest change in expression due to change in the 5 driver genes described above. These downstream genes (referred to interchangeably as “downstream”, “monitoring”, “reporter”, or “downstream reporter” genes) may be useful to monitor the effects of therapy of COVID-19 ARDS by methods known in the art (e.g., qPCR, qRT-PCR, digital PCR, ELISA, and the like) using one or more driver genes as drug targets. These 5 downstream genes may be useful as drug targets themselves, as disclosed herein.

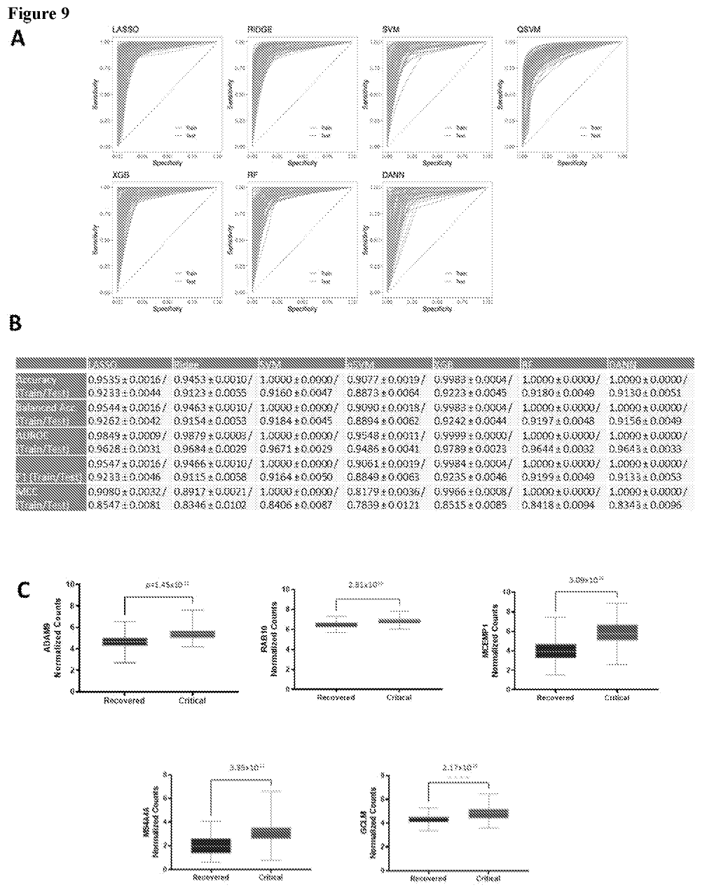

The usefulness of the 600 genes identified in this first group of patients was then evaluated in a second patient cohort, consisting of critical COVID-19 patients sampled at ICU entry and recovered critical patients sampled at three months after ICU exit. The top 600 genes from the first patient cohort were able to significantly differentiate between critical and recovered patients (FIGS. 9A, 9B, and Table 5); classification performance when training on the differentially expressed genes between critical and recovered patients is nearly the same (not shown), indicating the high degree of generalizability of this gene signature. Moreover, the five identified driver genes in patient cohort 1 were also shown to be up-regulated in critical patients in this second patient cohort (FIG. 9C). Accordingly, it will be appreciated by those of skill in the art that the gene signature, i.e., the genes set forth in Table 5, may be used in place of, or in addition to, genes ADAM9, MCEMP1, MS4A4A, RAB10, GCLM, EPHX2, RORA, CFAP97, ARL4C or ACSS1 in the the methods disclosed herein. Purely for the purpose of exemplification, one of skill in the art will understand that the methods disclosed herein may comprise one or more of the steps of (a) identifying from the sequencing of said sample at least one single-nucleotide polymorphism (SNP) in one or more of genes set forth in Table 5; (b) measuring the level of soluble protein expressed by one or more of the genes set forth in Table 5 in a sample from the subject; (c) measuring the expression level of one or more of the genes set forth in Table 5 at the RNA level in a sample from the subject; and/or (d) measuring the expression level of one or more of the genes set forth in Table 5 at the protein level in a sample from the subject.

TABLE 5
Top 600 genes

| EnsID | Gene name | EnsID | Gene name |
|---|---|---|---|
| ENSG00000234851 | RPL23AP42 | ENSG00000272617 | COG8 |
| ENSG00000112290 | WASF1 | ENSG00000240875 | LINC00886 |
| ENSG00000213553 | RPLPOP6 | ENSG00000089220 | PEBP1 |
| ENSG00000213442 | RPL18AP3 | ENSG00000165685 | TMEM52B |
| ENSG00000236552 | RPL13AP5 | ENSG00000125656 | CLPP |
| ENSG00000134545 | KLRC1 | ENSG00000099910 | KLHL22 |
| ENSG00000242071 | RPL7AP6 | ENSG00000167967 | E4F1 |
| ENSG00000084734 | GCKR | ENSG00000067601 | PMS2P4 |
| ENSG00000183578 | TNFAIP8L3 | ENSG00000164828 | SUN1 |
| ENSG00000137869 | CYP19A1 | ENSG00000172057 | ORMDL3 |
| ENSG00000108950 | FAM20A | ENSG00000197930 | ERO1A |
| ENSG00000154734 | ADAMTS1 | ENSG00000106266 | SNX8 |
| ENSG00000218426 | RP11-475C16.1 | ENSG00000108953 | YWHAE |
| ENSG00000167792 | NDUFV1 | ENSG00000175352 | NRIP3 |
| ENSG00000226608 | FTLP3 | ENSG00000112031 | MTRF1L |
| ENSG00000211821 | TRDV2 | ENSG00000196230 | TUBB |
| ENSG00000168209 | DDIT4 | ENSG00000106789 | CORO2A |
| ENSG00000023909 | GCLM | ENSG00000204936 | CD177 |
| ENSG00000254893 | AC113404.1 | ENSG00000017260 | ATP2C1 |
| ENSG00000172531 | PPP1CA | ENSG00000185056 | C5orf47 |
| ENSG00000182489 | XKRX | ENSG00000106003 | LFNG |
| ENSG00000203896 | LIME1 | ENSG00000231027 | AC079325.6 |
| ENSG00000196205 | EEF1A1P5 | ENSG00000183444 | OR7E38P |
| ENSG00000167105 | TMEM92 | ENSG00000214063 | TSPAN4 |
| ENSG00000182054 | IDH2 | ENSG00000108578 | BLMH |
| ENSG00000181090 | EHMT1 | ENSG00000163516 | ANKZF1 |
| ENSG00000100100 | PIK3IP1 | ENSG00000067057 | PFKP |
| ENSG00000036448 | MYOM2 | ENSG00000166401 | SERPINB8 |
| ENSG00000197063 | MAFG | ENSG00000092200 | RPGRIP1 |
| ENSG00000105193 | RPS16 | ENSG00000162775 | RBM15 |
| ENSG00000229638 | RPL4P4 | ENSG00000159618 | ADGRG5 |
| ENSG00000109472 | CPE | ENSG00000247315 | ZCCHC3 |
| ENSG00000167658 | EEF2 | ENSG00000074966 | TXK |
| ENSG00000183019 | MCEMP1 | ENSG00000105607 | GCDH |
| ENSG00000105373 | GLTSCR2 | ENSG00000142208 | AKT1 |
| ENSG00000225231 | AC091814.2 | ENSG00000111670 | GNPTAB |
| ENSG00000167680 | SEMA6B | ENSG00000126602 | TRAP1 |
| ENSG00000007264 | MATK | ENSG00000135643 | KCNMB4 |
| ENSG00000211829 | TRDC | ENSG00000228300 | C19orf24 |
| ENSG00000234797 | RPS3AP6 | ENSG00000281852 | LINC00891 |
| ENSG00000170439 | METTL7B | ENSG00000063978 | RNF4 |
| ENSG00000181201 | HIST3H2BA | ENSG00000184557 | SOCS3 |
| ENSG00000132613 | MTSS1L | ENSG00000130590 | SAMD10 |
| ENSG00000156265 | MAP3K7CL | ENSG00000155158 | TTC39B |
| ENSG00000165092 | ALDH1A1 | ENSG00000077684 | JADE1 |
| ENSG00000103415 | HMOX2 | ENSG00000187837 | HIST1H1C |
| ENSG00000080546 | SESN1 | ENSG00000211689 | TRGC1 |
| ENSG00000141736 | ERBB2 | ENSG00000241258 | CRCP |
| ENSG00000010810 | FYN | ENSG00000136830 | FAM129B |
| ENSG00000124575 | HIST1H1D | ENSG00000005022 | SLC25A5 |
| ENSG00000254415 | SIGLEC14 | ENSG00000179222 | MAGED1 |
| ENSG00000253190 | AC084082.3 | ENSG00000272540 | XXbac-BPG252P9.9 |
| ENSG00000178952 | TUFM | ENSG00000105568 | PPP2R1A |
| ENSG00000099622 | CIRBP | ENSG00000084733 | RAB10 |
| ENSG00000172053 | QARS | ENSG00000196218 | RYR1 |
| ENSG00000120262 | CCDC170 | ENSG00000146243 | IRAK1BP1 |
| ENSG00000137441 | FGFBP2 | ENSG00000198929 | NOS1AP |
| ENSG00000136710 | CCDC115 | ENSG00000116977 | LGALS8 |
| ENSG00000230071 | RPL4P6 | ENSG00000143515 | ATP8B2 |
| ENSG00000068028 | RASSF1 | ENSG00000163041 | H3F3A |
| ENSG00000211695 | TRGV9 | ENSG00000172831 | CES2 |
| ENSG00000103769 | RAB11A | ENSG00000197912 | SPG7 |
| ENSG00000138031 | ADCY3 | ENSG00000132170 | PPARG |
| ENSG00000202058 | RN7SKP80 | ENSG00000134668 | SPOCD1 |
| ENSG00000169122 | FAM110B | ENSG00000167984 | NLRC3 |
| ENSG00000169252 | ADRB2 | ENSG00000087589 | CASS4 |
| ENSG00000007350 | TKTL1 | ENSG00000198932 | GPRASP1 |
| ENSG00000243244 | STON1 | ENSG00000183625 | CCR3 |
| ENSG00000054611 | TBC1D22A | ENSG00000162191 | UBXN1 |
| ENSG00000110321 | EIF4G2 | ENSG00000125520 | SLC2A4RG |
| ENSG00000213366 | GSTM2 | ENSG00000139697 | SBNO1 |
| ENSG00000277972 | CISD3 | ENSG00000198821 | CD247 |
| ENSG00000130414 | NDUFA10 | ENSG00000173917 | HOXB2 |
| ENSG00000169727 | GPS1 | ENSG00000115232 | ITGA4 |
| ENSG00000150594 | ADRA2A | ENSG00000197457 | STMN3 |
| ENSG00000100316 | RPL3 | ENSG00000078124 | ACER3 |
| ENSG00000119714 | GPR68 | ENSG00000158435 | CNOT11 |
| ENSG00000105048 | TNNT1 | ENSG00000168685 | IL7R |
| ENSG00000149823 | VPS51 | ENSG00000205765 | C5orf51 |
| ENSG00000180096 | SEPT1 | ENSG00000177427 | MIEF2 |
| ENSG00000065268 | WDR18 | ENSG00000162591 | MEGF6 |
| ENSG00000166446 | CDYL2 | ENSG00000071462 | WBSCR22 |
| ENSG00000072134 | EPN2 | ENSG00000175106 | TVP23C |
| ENSG00000166394 | CYB5R2 | ENSG00000157881 | PANK4 |
| ENSG00000169045 | HNRNPH1 | ENSG00000153208 | MERTK |
| ENSG00000215021 | PHB2 | ENSG00000211451 | GNRHR2 |
| ENSG00000161381 | PLXDC1 | ENSG00000114841 | DNAH1 |
| ENSG00000170430 | MGMT | ENSG00000109084 | TMEM97 |
| ENSG00000161016 | RPL8 | ENSG00000137055 | PLAA |
| ENSG00000100823 | APEX1 | ENSG00000233476 | EEF1A1P6 |
| ENSG00000078043 | PIAS2 | ENSG00000246223 | LINC01550 |
| ENSG00000147403 | RPL10 | ENSG00000187091 | PLCD1 |
| ENSG00000171522 | PTGER4 | ENSG00000119688 | ABCD4 |
| ENSG00000038427 | VCAN | ENSG00000134954 | ETS1 |
| ENSG00000177239 | MAN1B1 | ENSG00000268173 | AC007192.4 |
| ENSG00000180739 | S1PR5 | ENSG00000132153 | DHX30 |
| ENSG00000064787 | BCAS1 | ENSG00000011485 | PPP5C |
| ENSG00000176978 | DPP7 | ENSG00000223972 | DDX11L1 |
| ENSG00000229473 | RGS17P1 | ENSG00000027075 | PRKCH |
| ENSG00000100450 | GZMH | ENSG00000165168 | CYBB |
| ENSG00000271447 | MMP28 | ENSG00000089916 | GPATCH2L |
| ENSG00000088682 | COQ9 | ENSG00000054654 | SYNE2 |
| ENSG00000067225 | PKM | ENSG00000198892 | SHISA4 |
| ENSG00000129103 | SUMF2 | ENSG00000141556 | TBCD |
| ENSG00000183049 | CAMK1D | ENSG00000163959 | SLC51A |
| ENSG00000163155 | LYSMD1 | ENSG00000164483 | SAMD3 |
| ENSG00000163346 | PBXIP1 | ENSG00000145555 | MYO10 |
| ENSG00000141002 | TCF25 | ENSG00000245080 | MIR3150B |
| ENSG00000110079 | MS4A4A | ENSG00000163249 | CCNYL1 |
| ENSG00000150630 | VEGFC | ENSG00000150764 | DIXDC1 |
| ENSG00000258227 | CLEC5A | ENSG00000152969 | JAKMIP1 |
| ENSG00000139572 | GPR84 | ENSG00000125457 | MIF4GD |
| ENSG00000095906 | NUBP2 | ENSG00000148803 | FUOM |
| ENSG00000184787 | UBE2G2 | ENSG00000167618 | LAIR2 |
| ENSG00000150687 | PRSS23 | ENSG00000084693 | AGBL5 |
| ENSG00000123689 | GOS2 | ENSG00000123096 | SSPN |
| ENSG00000147650 | LRP12 | ENSG00000152380 | FAM151B |
| ENSG00000170291 | ELP5 | ENSG00000077943 | ITGA8 |
| ENSG00000166289 | PLEKHF1 | ENSG00000213866 | YBX1P10 |
| ENSG00000109062 | SLC9A3R1 | ENSG00000037757 | MRI1 |
| ENSG00000133687 | TMTC1 | ENSG00000197409 | HIST1H3D |
| ENSG00000176974 | SHMT1 | ENSG00000171425 | ZNF581 |
| ENSG00000170425 | ADORA2B | ENSG00000211772 | TRBC2 |
| ENSG00000150938 | CRIM1 | ENSG00000144369 | FAM171B |
| ENSG00000204839 | MROH6 | ENSG00000011454 | RABGAP1 |
| ENSG00000137831 | UACA | ENSG00000130520 | LSM4 |
| ENSG00000143772 | ITPKB | ENSG00000081189 | MEF2C |
| ENSG00000136634 | IL10 | ENSG00000244038 | DDOST |
| ENSG00000170027 | YWHAG | ENSG00000139641 | ESYT1 |
| ENSG00000153531 | ADPRHL1 | ENSG00000127837 | AAMP |
| ENSG00000174600 | CMKLR1 | ENSG00000139636 | LMBR1L |
| ENSG00000126264 | HCST | ENSG00000277734 | TRAC |
| ENSG00000134590 | FAM127A | ENSG00000106701 | FSD1L |
| ENSG00000133561 | GIMAP6 | ENSG00000105223 | PLD3 |
| ENSG00000129038 | LOXL1 | ENSG00000171649 | ZIK1 |
| ENSG00000175390 | EIF3F | ENSG00000078098 | FAP |
| ENSG00000146540 | C7orf50 | ENSG00000136052 | SLC41A2 |
| ENSG00000187498 | COL4A1 | ENSG00000198242 | RPL23A |
| ENSG00000196876 | SCN8A | ENSG00000089053 | ANAPC5 |
| ENSG00000182621 | PLCB1 | ENSG00000135486 | HNRNPA1 |
| ENSG00000248487 | ABHD14A | ENSG00000160439 | RDH13 |
| ENSG00000233806 | LINC01237 | ENSG00000168778 | TCTN2 |
| ENSG00000168615 | ADAM9 | ENSG00000074071 | MRPS34 |
| ENSG00000213413 | PVRIG | ENSG00000144893 | MED12L |
| ENSG00000107175 | CREB3 | ENSG00000167526 | RPL13 |
| ENSG00000271383 | NBPF19 | ENSG00000100242 | SUN2 |
| ENSG00000270069 | MIR222HG | ENSG00000172215 | CXCR6 |
| ENSG00000198483 | ANKRD35 | ENSG00000100029 | PES1 |
| ENSG00000213626 | LBH | ENSG00000117868 | ESYT2 |
| ENSG00000100453 | GZMB | ENSG00000108107 | RPL28 |
| ENSG00000148335 | NTMT1 | ENSG00000145604 | SKP2 |
| ENSG00000164741 | DLC1 | ENSG00000103723 | AP3B2 |
| ENSG00000007312 | CD79B | ENSG00000185475 | TMEM179B |
| ENSG00000151012 | SLC7A11 | ENSG00000106686 | SPATA6L |
| ENSG00000204852 | TCTN1 | ENSG00000107742 | SPOCK2 |
| ENSG00000168246 | UBTD2 | ENSG00000160613 | PCSK7 |
| ENSG00000183734 | ASCL2 | ENSG00000137098 | SPAG8 |
| ENSG00000169093 | ASMTL | ENSG00000183077 | AFMID |
| ENSG00000169504 | CLIC4 | ENSG00000178115 | GOLGA8Q |
| ENSG00000159403 | C1R | ENSG00000067606 | PRKCZ |
| ENSG00000164070 | HSPA4L | ENSG00000110013 | SIAE |
| ENSG00000205138 | SDHAF1 | ENSG00000132386 | SERPINF1 |
| ENSG00000112667 | DNPH1 | ENSG00000152464 | RPP38 |
| ENSG00000113361 | CDH6 | ENSG00000122420 | PTGFR |
| ENSG00000157326 | DHRS4 | ENSG00000204628 | RACK1 |
| ENSG00000180251 | SLC9A4 | ENSG00000130600 | H19 |
| ENSG00000178028 | DMAP1 | ENSG00000182866 | LCK |
| ENSG00000224861 | YBX1P1 | ENSG00000143184 | XCL1 |
| ENSG00000177600 | RPLP2 | ENSG00000108298 | RPL19 |
| ENSG00000070404 | FSTL3 | ENSG00000042832 | TG |
| ENSG00000134765 | DSC1 | ENSG00000226777 | KIAA0125 |
| ENSG00000111696 | NT5DC3 | ENSG00000105376 | ICAM5 |
| ENSG00000138685 | FGF2 | ENSG00000196329 | GIMAP5 |
| ENSG00000149182 | ARFGAP2 | ENSG00000136160 | EDNRB |
| ENSG00000198586 | TLK1 | ENSG00000145982 | FARS2 |
| ENSG00000105640 | RPL18A | ENSG00000170962 | PDGFD |
| ENSG00000136999 | NOV | ENSG00000196405 | EVL |
| ENSG00000165457 | FOLR2 | ENSG00000100024 | UPB1 |
| ENSG00000177830 | CHID1 | ENSG00000073111 | MCM2 |
| ENSG00000200488 | RN7SKP203 | ENSG00000140988 | RPS2 |
| ENSG00000141560 | FN3KRP | ENSG00000055950 | MRPL43 |
| ENSG00000174837 | ADGRE1 | ENSG00000188042 | ARL4C |
| ENSG00000275379 | HIST1H3I | ENSG00000219529 | AP000580.1 |
| ENSG00000053254 | FOXN3 | ENSG00000223865 | HLA-DPB1 |
| ENSG00000122741 | DCAF10 | ENSG00000272886 | DCP1A |
| ENSG00000004455 | AK2 | ENSG00000213203 | GIMAP1 |
| ENSG00000104660 | LEPROTL1 | ENSG00000155657 | TTN |
| ENSG00000123933 | MXD4 | ENSG00000071909 | MYO3B |
| ENSG00000152760 | TCTEX1D1 | ENSG00000197646 | PDCD1LG2 |
| ENSG00000042493 | CAPG | ENSG00000145912 | NHP2 |
| ENSG00000069998 | CECR5 | ENSG00000001630 | CYP51A1 |
| ENSG00000169991 | IFFO2 | ENSG00000231389 | HLA-DPA1 |
| ENSG00000233901 | LINC01503 | ENSG00000127152 | BCL11B |
| ENSG00000274290 | HIST1H2BE | ENSG00000063177 | RPL18 |
| ENSG00000022556 | NLRP2 | ENSG00000206561 | COLQ |
| ENSG00000128185 | DGCR6L | ENSG00000181036 | FCRL6 |
| ENSG00000198574 | SH2D1B | ENSG00000175970 | UNC119B |
| ENSG00000168229 | PTGDR | ENSG00000069667 | RORA |
| ENSG00000234585 | CCT6P3 | ENSG00000134627 | PIWIL4 |
| ENSG00000112514 | CUTA | ENSG00000164053 | ATRIP |
| ENSG00000138796 | HADH | ENSG00000205609 | EIF3CL |
| ENSG00000122140 | MRPS2 | ENSG00000006015 | C19orf60 |
| ENSG00000230124 | ACBD6 | ENSG00000174080 | CTSF |
| ENSG00000183691 | NOG | ENSG00000095383 | TBC1D2 |
| ENSG00000072736 | NFATC3 | ENSG00000124181 | PLCG1 |
| ENSG00000213071 | LPAL2 | ENSG00000178146 | RP1-232L22_B.1 |
| ENSG00000105671 | DDX49 | ENSG00000111371 | SLC38A1 |
| ENSG00000187024 | PTRH1 | ENSG00000244682 | FCGR2C |
| ENSG00000152256 | PDK1 | ENSG00000115085 | ZAP70 |
| ENSG00000183828 | NUDT14 | ENSG00000115687 | PASK |
| ENSG00000102893 | PHKB | ENSG00000140968 | IRF8 |
| ENSG00000158006 | PAFAH2 | ENSG00000127554 | GFER |
| ENSG00000250565 | ATP6V1E2 | ENSG00000224631 | RP11-5106.1 |
| ENSG00000166997 | CNPY4 | ENSG00000228960 | OR2A9P |
| ENSG00000235655 | H3F3AP4 | ENSG00000120915 | EPHX2 |
| ENSG00000161618 | ALDH16A1 | ENSG00000137818 | RPLP1 |
| ENSG00000134901 | KDELC1 | ENSG00000011478 | QPCTL |
| ENSG00000104490 | NCALD | ENSG00000139193 | CD27 |
| ENSG00000109436 | TBC1D9 | ENSG00000153283 | CD96 |
| ENSG00000108443 | RPS6KB1 | ENSG00000269335 | IKBKG |
| ENSG00000143167 | GPA33 | ENSG00000120705 | ETF1 |
| ENSG00000267737 | AC061992.2 | ENSG00000112333 | NR2E1 |
| ENSG00000164081 | TEX264 | ENSG00000102531 | FNDC3A |
| ENSG00000079691 | LRRC16A | ENSG00000138821 | SLC39A8 |
| ENSG00000165060 | FXN | ENSG00000161179 | YDJC |
| ENSG00000173114 | LRRN3 | ENSG00000197043 | ANXA6 |
| ENSG00000119042 | SATB2 | ENSG00000152270 | PDE3B |
| ENSG00000186594 | MIR22HG | ENSG00000101158 | NELFCD |
| ENSG00000109790 | KLHL5 | ENSG00000068400 | GRIPAP1 |
| ENSG00000162076 | FLYWCH2 | ENSG00000128524 | ATP6V1F |
| ENSG00000159692 | CTBP1 | ENSG00000263464 | PPIAL4C |
| ENSG00000178386 | ZNF223 | ENSG00000166529 | ZSCAN21 |
| ENSG00000229689 | AC009237.8 | ENSG00000164323 | CFAP97 |
| ENSG00000149294 | NCAM1 | ENSG00000189319 | FAM53B |
| ENSG00000169100 | SLC25A6 | ENSG00000137941 | TTLL7 |
| ENSG00000148303 | RPL7A | ENSG00000122971 | ACADS |
| ENSG00000168175 | MAPK1IP1L | ENSG00000122861 | PLAU |
| ENSG00000095203 | EPB41L4B | ENSG00000141499 | WRAP53 |
| ENSG00000172164 | SNTB1 | ENSG00000130811 | EIF3G |
| ENSG00000123119 | NECAB1 | ENSG00000189420 | ZFP92 |
| ENSG00000135999 | EPC2 | ENSG00000135905 | DOCK10 |
| ENSG00000196562 | SULF2 | ENSG00000226380 | MIR29A |
| ENSG00000124942 | AHNAK | ENSG00000115306 | SPTBN1 |
| ENSG00000152684 | PELO | ENSG00000204287 | HLA-DRA |
| ENSG00000091428 | RAPGEF4 | ENSG00000239382 | ALKBH6 |
| ENSG00000116221 | MRPL37 | ENSG00000181991 | MRPS11 |
| ENSG00000243789 | JMJD7 | ENSG00000180871 | CXCR2 |
| ENSG00000272602 | ZNF595 | ENSG00000128791 | TWSG1 |
| ENSG00000262919 | FAM58A | ENSG00000063046 | EIF4B |
| ENSG00000108587 | GOSR1 | ENSG00000152234 | ATP5A1 |
| ENSG00000163251 | FZD5 | ENSG00000213015 | ZNF580 |
| ENSG00000101439 | CST3 | ENSG00000198034 | RPS4X |
| ENSG00000136068 | FLNB | ENSG00000148362 | C9orf142 |
| ENSG00000040933 | INPP4A | ENSG00000136156 | ITM2B |
| ENSG00000068724 | TTC7A | ENSG00000089737 | DDX24 |
| ENSG00000115523 | GNLY | ENSG00000130787 | HIP1R |
| ENSG00000130513 | GDF15 | ENSG00000163958 | ZDHHC19 |
| ENSG00000110934 | BIN2 | ENSG00000122188 | LAX1 |
| ENSG00000177570 | SAMD12 | ENSG00000154930 | ACSS1 |
| ENSG00000185897 | FFAR3 | ENSG00000156831 | NSMCE2 |
| ENSG00000115738 | ID2 | ENSG00000090382 | LYZ |
| ENSG00000196781 | TLE1 | ENSG00000154102 | C16orf74 |
| ENSG00000196415 | PRTN3 | ENSG00000154814 | OXNAD1 |
| ENSG00000100784 | RPS6KA5 | ENSG00000162910 | MRPL55 |
| ENSG00000183837 | PNMA3 | ENSG00000169592 | INO80E |
| ENSG00000129968 | ABHD17A | ENSG00000197506 | SLC28A3 |
| ENSG00000099985 | OSM | ENSG00000137571 | SLCO5A1 |
| ENSG00000135390 | ATP5G2 | ENSG00000228775 | WEE2-AS1 |
| ENSG00000134539 | KLRD1 | ENSG00000143799 | PARP1 |
| ENSG00000130783 | CCDC62 | ENSG00000100298 | APOBEC3H |
| ENSG00000104679 | R3HCC1 | ENSG00000147457 | CHMP7 |
| ENSG00000173812 | EIF1 | ENSG00000131378 | RFTN1 |
| ENSG00000128965 | CHAC1 | ENSG00000171658 | RP11-443P15.2 |
| ENSG00000073861 | TBX21 | ENSG00000178752 | ERFE |
| ENSG00000152952 | PLOD2 | ENSG00000178229 | ZNF543 |
| ENSG00000132967 | HMGB1P5 | ENSG00000113263 | ITK |
| ENSG00000175463 | TBC1D10C | ENSG00000237484 | AP000476.1 |
| ENSG00000196839 | ADA | ENSG00000129292 | PHF20L1 |
| ENSG00000161944 | ASGR2 | ENSG00000110063 | DCPS |
| ENSG00000085662 | AKR1B1 | ENSG00000197471 | SPN |
| ENSG00000162407 | PLPP3 | ENSG00000124177 | CHD6 |
| ENSG00000198890 | PRMT6 | ENSG00000171860 | C3AR1 |
| ENSG00000133138 | TBC1D8B | ENSG00000108465 | CDK5RAP3 |
| ENSG00000253522 | MIR3142HG | ENSG00000110448 | CD5 |
| ENSG00000166979 | EVA1C | ENSG00000019582 | CD74 |
| ENSG00000145287 | PLAC8 | ENSG00000186281 | GPAT2 |
| ENSG00000238121 | LINC00426 | ENSG00000137133 | HINT2 |
| ENSG00000148832 | PAOX | ENSG00000149016 | TUT1 |
| ENSG00000179921 | GPBAR1 | ENSG00000136717 | BIN1 |
| ENSG00000166707 | ZCCHC18 | ENSG00000178075 | GRAMD1C |
| ENSG00000235609 | AF127936.9 | ENSG00000010610 | CD4 |
| ENSG00000154767 | XPC | ENSG00000254772 | EEF1G |
| ENSG00000167107 | ACSF2 | ENSG00000099194 | SCD |
| ENSG00000197128 | ZNF772 | ENSG00000135736 | CCDC102A |
| ENSG00000131408 | NR1H2 | ENSG00000010165 | METTL13 |
| ENSG00000074964 | ARHGEF10L | ENSG00000133597 | ADCK2 |
| ENSG00000048028 | USP28 | ENSG00000226711 | FAM66C |
| ENSG00000105501 | SIGLEC5 | ENSG00000144445 | KANSL1L |
| ENSG00000106366 | SERPINE1 | ENSG00000107018 | RLN1 |
| ENSG00000113300 | CNOT6 | ENSG00000161405 | IKZF3 |

### Example 7: ADAM9 is a Major Driver of ARDS in Critical COVID-19 Patients

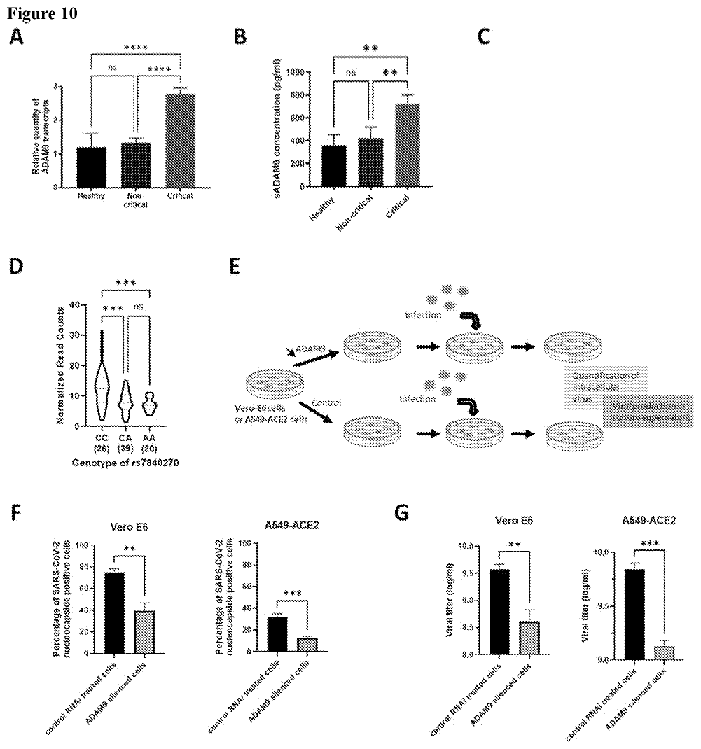

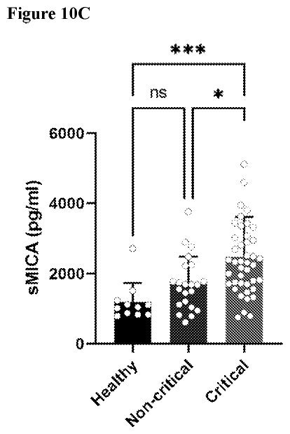

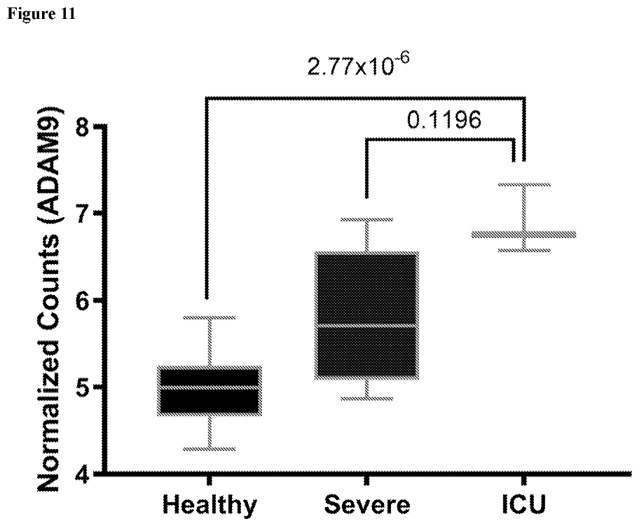

Among the five driver genes identified by structural causal modeling, focus was on experimentally determining the role of ADAM9 (A disintegrin and a metalloprotease) in COVID-19 etiology as (i) it is the gene with the greatest degree of causal influence in the SCM DAG, (ii) it is the only driver gene that has previously been shown to interact with SARS-CoV-2 by a global interactomics approach (Gordon et al., 2020a, 2020b) and (iii) it is an entry factor for another RNA virus, the Encephalomyocarditis Virus (Bazzone et al., 2019). ADAM9 is a metalloprotease with various functions that are either mediated by its disintegrin domain for adhesion or by its metalloprotease domain for the shedding of a large range of cell surface proteins (Chou et al., 2020). The ADAM9 gene encodes two isoforms encoding respectively for a membrane bound and a secreted protein. Although neither isoform could be detected by the proteomics approach, ADAM9 was up-regulated at the RNA level and the secreted form showed a higher concentration in the plasma of critical versus non-critical patients (FIGS. 10A and 10B). The transcriptional up-regulation of ADAM9 was also associated with disease severity in a previously published bulk RNA-seq dataset (FIG. 11) (Arunachalam et al., 2020). To assess a potential increased metalloprotease activity in the critical group, ELISA was used to quantify the soluble form of the MICA protein, which is known to be cleaved by ADAM9 (Kohga et al., 2010). The concentration of soluble MICA was indeed significantly higher in the plasma of critical patients as compared to non-critical patients and healthy controls (FIG. 10C). Global eQTL analysis using whole genome sequencing and RNA-seq data showed 8 SNPs associated with three of the top five putative driver genes with genome-wide significance (Table 6).

TABLE 6
eQTLs identified in three driver genes using MatrixeQTL

| SNP* | rs number | gene | beta | t-stat | P-value | FDR |
|---|---|---|---|---|---|---|
| chr8:38996464-C-A | rs7840270 | ADAM9 | −0.560481 | −4.461647 | 0.000034 | 0.038072 |
| chr8:38997543-G-T | rs7831735 | ADAM9 | −0.565580 | −4.359599 | 0.000049 | 0.046521 |
| chr19:7742229-G-A | rs11465401 | MCEMP1 | 1.912424 | 4.333792 | 0.000054 | 0.048775 |
| chr19:7742364-G-A | rs11465397 | MCEMP1 | 1.912424 | 4.333792 | 0.000054 | 0.048775 |
| chr11:60510522-C-T | rs189755275 | MS4A4A | 2.328040 | 4.358676 | 0.000049 | 0.046648 |
| chr11:60547398-G-A | rs76847438 | MS4A4A | 2.328040 | 4.358676 | 0.000049 | 0.046648 |
| chr11:60582964-G-A | rs10736707 | MS4A4A | −2.328040 | −4.358676 | 0.000049 | 0.046648 |
| chr11:60623519-G-A | rs10792287 | MS4A4A | −2.328040 | −4.358676 | 0.000049 | 0.046648 |

*positions refer to GRCh38

Among those, rs7840270 is localized just 0.3 kb upstream of the ADAM9 gene and an eQTL for blood expression reported in GTEX (FIG. 10D). In accordance with the observed up-regulation of the gene, the higher expressing allele C was more frequent in critical than in non-critical patients (71.3% vs. 50%, OR=2.48, P=0.017).

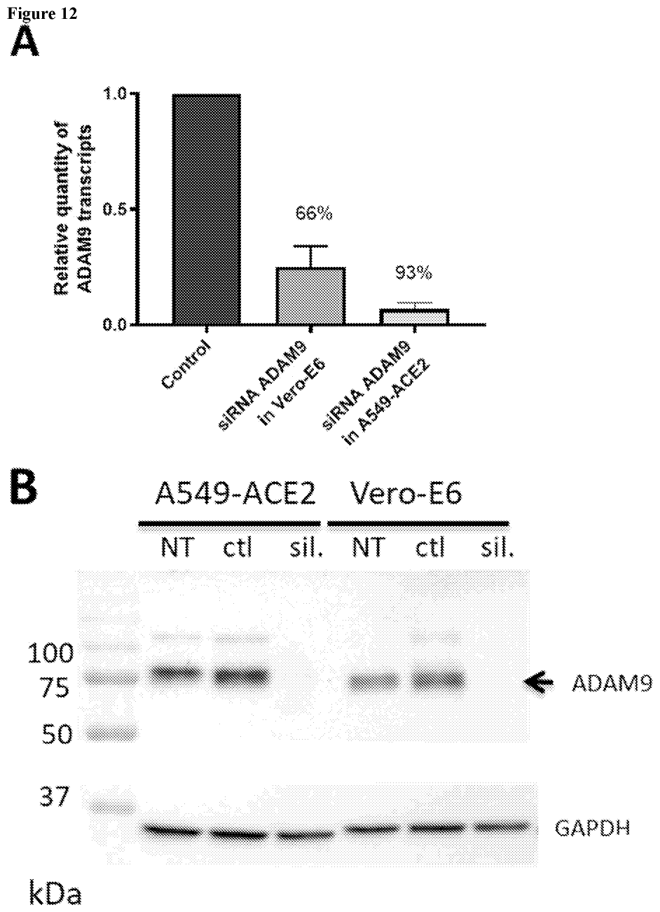

To assess the role of ADAM9 in viral infection, an in vitro assay was designed in which ADAM9 was silenced by siRNA in Vero-E6 or A549-ACE2 (Buchrieser et al., 2020) cells and subsequently infected the cells with SARS-CoV-2. Viral entry was monitored by flow cytometry quantification of the internalized nucleocapsid protein and the viral replication by quantitative viral RT-PCR in the culture supernatant (FIG. 10E). The average silencing efficiency reached 66% in vero-E6 cells and 93% in A549-ACE2 cells (FIG. 12). In both cell lines, the amount of internalized virus and the quantity of produced virus was significantly lower when ADAM9 was silenced as compared to the control condition that was treated with a scrambled siRNA (FIGS. 10F and 10G). This result indicates that ADAM9, which was an up-regulated in vivo driver in critical patients, facilitates viral infection and replication.

### Example 8: Discussion

A multi-omics strategy associated with integrated AI/ML and probabilistic programming methods was used to identify pathways and signatures that can differentiate critical from non-critical patients in a population of patients below 50 years of age and without major comorbidities. This in silico strategy provided a detailed view of the systemic immune response that was globally in line with previously published data. A consistent transcriptomic signature that was able to robustly differentiate critical from non-critical patients, as shown by the classification performance metrics assessed was also defined (FIG. 7A and Table 4). Notably, this signature can be generalized as the classification performance was shown to perform equally well in a replication cohort composed of 81 critically ill patients and 73 recovered critical patients (FIG. 9).

Using the top 600 gene expression features of the signature as the input for structural causal modeling, a causal network was derived, which uncovered five putative driver genes: RAB10, MCEMP1, MS4A4A, GCLM and ADAM9. RAB10 (Ras-related protein Rab-10) is a small GTPase that regulates macropynocytosis in phagocytes (Liu et al., 2020), a mechanism that has been suggested to be involved in SARS-CoV-2 entry in respiratory epithelial cells (Glebov, 2020). MCEMP1 (Mast Cell Expressed Membrane Protein 1) is a membrane protein specifically associated with lung mast cells and for which a lowered expression has been shown to reduce inflammation of septic mice (Li et al., 2005; Xie et al., 2020). MS4A4A (a member of the membrane-spanning, four domain family, subfamily A) is a surface marker for M2 macrophages which mediate immune responses in pathogen clearance (Sanyal et al., 2017) and regulates arginase 1 induction during macrophage polarization and lung inflammation in mice (Sui and Zeng, 2020). GCLM (Glutamate-Cysteine Ligase Modifier Subunit) is the first rate limiting enzyme of glutathione synthesis, a molecule that has been linked to severe COVID-19 (Sui and Zeng, 2020). ADAM9 (Disintegrin and metalloproteinase domain-containing protein 9), a metalloprotease with associated with a variety biological functions was made the focus of functional validations. The confirmed up-regulation at the RNA and protein levels in critical patients, the increased metalloprotease activity in these same patients, and ex vivo validation of its effect on viral uptake/replication are indeed strong arguments in favor of a possible therapeutic targeting of the protein to treat or prevent severe COVID-19.

Detailed multi-omics investigation in a well-characterized young, previously health-critical COVID-19 patient series, compared to non-critical patients, uncovered a landscape of blood molecular changes in critical patients. What is more, provided herein is a completely data-driven in silico AI/ML strategy, which was devoid of a priori biological information to provide potential candidate therapeutic targets that might be helpful in the ongoing battle against the COVID-19 pandemic. For example, though ADAM9 is the subject of cancer research, e.g., as a target for antibody-drug-conjugate therapy of solid tumors (Sui and Zeng, 2020), the data provided herein suggests a repurposing strategy using ADAM9 blocking antibodies or other therapeutic agents to reduce ADAM9 levels or activity to treat critical COVID-19 patients.

### Machine Learning Components

In some embodiments discussed above, a feature vector is provided to a trained classifier. In some embodiments, the learning system is pre-trained using training data. In some embodiments training data is retrospective data. In some embodiments, the retrospective data is stored in a data store. In some embodiments, the learning system may be additionally trained through manual curation of previously generated outputs. It will be appreciated that in addition to the specific examples provided above, a variety of other classifiers are suitable for use according to the present disclosure, including random decision forests, linear classifiers, support vector machines (SVM), and neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### INCORPORATION BY REFERENCE

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

A number of embodiments of the invention have been described. Nevertheless, it will be understood that various modifications may be made without departing from the spirit and scope of the invention. Accordingly, other embodiments are within the scope of the following claims. Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

1. A method for treating or preventing severe coronavirus disease 2019 (COVID-19) in a subject, comprising administering to the subject a composition comprising a modulating agent that decreases or increases the expression or gene product activity of one or more of ADAM9, MCEMP1, MS4A4A, RAB10, GCLM, EPHX2, RORA, CFAP97, ARL4C, and/or ACSS1 gene.

2. The method of claim 1, comprising the steps of:
(a) sequencing at least part of the subject's genome in a sample from said subject, wherein the at least part of said genome comprises one or more of an ADAM9, MCEMP1, MS4A4A, RAB10, GCLM, EPHX2, RORA, CFAP97, ARL4C, or ACSS1 gene;
(b) identifying from the sequencing of said sample at least one single-nucleotide polymorphism (SNP) in one or more of genes: ADAM9, MCEMP1, MS4A4A, RAB10, GCLM, EPHX2, RORA, CFAP97, ARL4C, or ACSS1; and
(c) administering a corresponding modulating agent that decreases or increases the expression or activity of the gene products of one or more of ADAM9, MCEMP1, MS4A4A, RAB10, GCLM, EPHX2, RORA, CFAP97, ARL4C, or ACSS1.

3-6. (canceled)

7. The method of claim 2 or 25, wherein the SNP is rs7840270, rs7831735, rs11465401, rs11465397, rs11465397, rs189755275, rs76847438, rs10736707, or rs10792287.

8. The method of claim 1, comprising the steps of:
(a) sequencing and/or measuring at least part of the subject's transcriptome in a sample from said subject, wherein the at least part of said transcriptome comprises at least one mRNA of ADAM9, MCEMP1, MS4A4A, RAB10, GCLM, EPHX2, RORA, CFAP97, ARL4C, or ACSS1 genes;
(b) determining the expression level of at least one of ADAM9, MCEMP1, MS4A4A, RAB10, GCLM, EPHX2, RORA, CFAP97, ARL4C, and/or ACSS1 in step (a) and comparing it to a reference value, wherein the expression level of at least one of ADAM9, MCEMP1, MS4A4A, RAB10, GCLM, EPHX2, RORA, CFAP97, ARL4C, or ACSS1 gene relative to the reference value indicates whether the subject will respond to a corresponding modulating agent that decreases or increases the expression or activity of the gene products of ADAM9, MCEMP1, MS4A4A, RAB10, GCLM, EPHX2, RORA, CFAP97, ARL4C, and/or ACSS1 genes.

9-12. (canceled)

13. A method for monitoring a human subject suffering from CoVID-19 for potential treatment with a modulating agent that decreases or increases the expression or activity of the gene products of one or more of ADAM9, MCEMP1, MS4A4A, RAB10, GCLM, EPHX2, RORA, CFAP97, ARL4C, or ACSS1, comprising obtaining a sample from the subject at predetermined intervals;
a) obtaining a gene expression profile from the sample, wherein the expression profile comprises expression levels for one or more genes; wherein said one or more genes comprises at least ADAM9, MCEMP1, MS4A4A, RAB10, GCLM, EPHX2, RORA, CFAP97, ARL4C, or ACSS1; and
b) comparing the gene expression profile of each sample chronologically, wherein an increase in one or more of ADAM9, MCEMP1, MS4A4A, RAB10, GCEM, EPHX2, RORA, CFAP97, ARL4C, or ACSS1 expression over time identifies the subject as a critical subject.

14. The method of any one of claims 1, 2, 8, 13, and 25, wherein the modulating agent is an inhibitor of the expression or activity of the gene product, a small molecule, or an antibody inhibitor of ADAM9 expression and/or activity.

15. The method of claim 14, wherein the inhibitor is an interfering nucleic acid specific for the mRNA product of at least ADAM9 gene.

16. The method of claim 15, wherein the interfering nucleic acid is a siRNA, shRNA, miRNA, or peptide nucleic acid (PNA).

17. The method of claim 15, wherein the interfering nucleic acid is HSS112867.

18-24. (canceled)

25. A method for predicting the likelihood of a subject infected with SARS-CoV-2 progressing to severe COVID-19, comprising the steps of:
(a) sequencing or genotyping of at least part of the subject's genome in a sample from said subject, wherein the at least part of said genome comprises one or more of an ADAM9, MCEMP1, MS4A4A, RAB10, GCLM, EPHX2, RORA, CFAP97, ARL4C or ACSS1 gene;
(b) identifying from the sequencing or genotyping of said sample at least one SNP in one or more of genes ADAM9, MCEMP1, MS4A4A, RAB10, GCLM, EPHX2, RORA, CFAP97, ARL4C, or ACSSP, and
(c) using individual SNPs to form individual SNP risk or to combine multiple SNPs to define polygenic risk scores to provide an indication of the likelihood of progression to severe COVID-19.

26. A method for predicting the likelihood of a subject infected with SARS-CoV-2 to progressing to severe COVID-19, comprising the steps of:
(a) sequencing or genotyping at least part of the subject's genome in a sample from said subject, or sequencing or other measurement or measuring of at least part of the subject's transcriptome in a sample from said subject, wherein the at least part of said genome or transcriptome comprises one or more of an ADAM9, MCEMP1, MS4A4A, RAB10, GCLM, EPHX2, RORA, CFAP97, ARL4C or ACSS1 gene;
(b) identifying from the sequencing or genotyping of said sample at least one SNP in one or more of genes ADAM9, MCEMP1, MS4A4A, RAB10, GCLM, EPHX2, RORA, CFAP97, ARL4C, or ACSSL, or determining the expression level of at least one of ADAM9, MCEMP1, MS4A4A, RAB10, GCLM, EPHX2, RORA, CFAP97, ARL4C, or ACSS1 of step (a):
(c) forming from said at least one SNP or from said expression level a feature vector, and
(d) providing the feature vector to a trained classifier and receiving therefrom an indication of the likelihood of progression to severe COVID-19.

27. (canceled)

28. The method of claim 26, wherein the trained classifier comprises a LASSO model, a ridge regression model, a support vector machine (SVM), a quantum support vector machine (qSVM), an XGBoost model (XGB) a random forest (RF), or a DANN artificial neural network.

29-31. (canceled)

32. The method of claim 25 or 26, wherein said method is a method for predicting the likelihood of a subject with respiratory symptoms or signs progressing to severe acute respiratory distress syndrome (ARDS) and initiating more aggressive or preventative treatment, comprising the additional steps of:
(a) sequencing of at least part of the subject's transcriptome in a sample from said subject, wherein the at least part of said transcriptome comprises at least 600 genes in a genomic signature;
(b) determining the expression levels of the at least 600 genes in the genomic signature;
(c) forming from said expression levels a feature vector, and
(d) providing the feature vector to a trained classifier and receiving therefrom an indication of the likelihood of progression to severe ARDS;
wherein the at least 600 genes comprises: RPL23AP42, COG8, WASFI, LINC00886, RPLP0P6, PEBPI, RPL18AP3, TMEM52B, RPL13AP5, CLPP, KLRCI, KLHL22, RPL7AP6, E4F1, GCKR, PMS2P4, TNFAIP8L3, SUNI, CYP19A1, ORMDL3, FAM20A, EROIA, ADAMTSI, SNX8, RPI1-475C16.1, YWHAE, NDUFVI, NRIP3, FTLP3, MTRFIL, TRDV2, TUBB, DDIT4, CORO2A, GCLM, CD177, AC113404.1, ATP2C1, PPPICA, C5orf47, XKRX, LFNG, LIMEI, AC079325.6, EEFIAIP5, OR7E38P, TMEM92, TSPAN4, IDH2, BLMH, EHMTI, ANKZFI, PIK.31P1, PFKP, MYOM2, SERPINB8, MAFG, RPGRIPI, RPS16, RBM15, RPL4P4, ADGRG5, CPE, ZCCHC3, EEF2, TXK, MCEMP1, GCDH, GLTSCR2, AKTI, AC091814.2, GNPTAB, SEMA6B, TRAPI, MATK, KCNMB4, TRDC, Cl9orf24, RPS3AP6, LINC00891, METTL7B, RNF4, HIST3H2BA, SOCS3, MTSSIL, SAMDI0, MAP3K7CL, TTC39B, ALDHIAI, JADEI, HMOX2, HISTIHIC, SESNI, TRGCI, ERBB2, CRCP, FYN, FAM129B, HISTIHID, SLC25A5, SIGLEC14, MAGED1, AC084082.3, XXbac-BPG252P9.9, TUFM, PPP2RIA, CIRBP, RAB10, OARS, RYRI, CCDC170, IRAKIBPI, FGFBP2, NOSIAP, CCDC115, LGALS8, RPL4P6, ATP8B2, RASSFI, H3F3A, TRGV9, CES2, RAB11A, SPG7, ADCY3, PPARG, RN7SKP80, SPOCDI, FAM110B, NLRC3, ADRB2, CASS4, TKTLI, GPRASPI, STONI, CCR3, TBCID22A, UBXNI, EIF4G2, SLC2A4RG, GSTM2, SBNOI, CISD3, CD247, NDUFAIO, HOXB2, GPS1, ITGA4, ADRA2A, STMN3, RPL3, ACER3, GPR68, CNOT11, TNNTI, IL7R, VPS51, C5orf51, SEPTI, MIEF2, WDR18, MEGF6, CDYL2, WBSCR22, EPN2, TVP23C, CYB5R2, PANK4, HNRNPHI, MERTK, PHB2, GNRHR2, PLXDCI, DNAHI, MGMT, TMEM97, RPL8, PLAA, APEXI, EEFIAIP6, PIAS2, LINC01550, RPLI0, PLCDI, PTGER4, ABCD4, VCAN, ETSI, MANIBI, AC007192.4, SIPR5, DHX30, BCASI, PPP5C, DPP7, DDXIILL, RGS17P1, PRKCH, GZMH, CYBB, MMP28, GPATCH2L, COQ9, SYNE2, PKM, SHISA4, SUMF2, TBCD, CAMKID, SLC51A, LYSMDI, SAMD3, PBXIPI, MYOI0, TCF25, MIR3150B, MS4A4A, CCNYLI, VEGFC, DIXDCI, CLEC5A, JAKMIPI, GPR84, MIF4GD, NUBP2, FUOM, UBE2G2, LAIR2, PRSS23, AGBL5, G0S2, SSPN, LRP12, FAM151B, ELP5, ITGA8, PLEKHFI, YBXIPI0, SLC9A3R1, MRII, TMTCI, HISTIH3D, SHMTI, ZNF581, ADORA2B, TRBC2, CRIMI, FAM171B, MROH6, RABGAPI, UACA, LSM4, ITPKB, MEF2C, ILI0, DDOST, YWHAG, ESYTI, ADPRHLI, AAMP, CMKLRI, LMBRIL, HCST, TRAC, FAM127A, FSDIL, GIMAP6, PLD3, LOXLI, ZIKI, EIF3F, FAP, C7orf50, SLC41A2, COL4A1, RPL23A, SCN8A, ANAPC5, PLCBI, HNRNPAI, ABHD14A, RDH13, LINC01237, TCTN2, ADAM9, MRPS34, PVRIG, MEDI2L, CREB3, RPL13, NBPF19, SUN2, MIR222HG, CXCR6, ANKRD35, PESI, LBH, ESYT2, GZMB, RPL28, NTMTI, SKP2, DLCI, AP3B2, CD79B, TMEM179B, SLC7A11, SPATA6L, TCTNI, SPOCK2, UBTD2, PCSK7, ASCL2, SPAG8, ASMTL, AFMID, CLIC4, GOLGA8Q, CIR, PRKCZ, HSPA4L, SIAE, SDHAFI, SERPINFI, DNPHI, RPP38, CDH6, PTGFR, DHRS4, RACKI, SLC9A4, H19, DMAPI, LCK, YBXIPI, XCLI, RPLP2, RPL19, FSTL3, TG, DSCI, KIAA0125, NT5DC3, ICAM5, FGF2, GIMAP5, ARFGAP2, EDNRB, TLKI, FARS2, RPL18A, PDGFD, NOV, EVL, FOLR2, UPBI, CHIDI, MCM2, RN7SKP203, RPS2, FN3KRP, MRPL43, ADGREI, ARL4C, HISTIH3I, AP000580.1, FOXN3, HLA-DPBI, DCAFI0, DCPIA, AK2, GIMAP1, LEPROTL1, TTN, MXD4, MYO3B, TCTEX1D1, PDCD1 LG2, CAPG, NHP2, CECR5, CYP51A1, IFFO2, HLA-DPA1, LINC01503, BCL11B, HIST1H2BE, RPL18, NLRP2, COLQ, DGCR6L, FCRL6, SH2D1B, UNC119B, PTGDR, RORA, CCT6P3, PIWIL4, CUTA, ATRIP, HADH, EIF3CL, MRPS2, C19orf60, ACBD6, CTSF, NOG, TBC1D2, NFATC3, PLCG1, LPAL2, RP1-232L22B.1, DDX49, SLC38A1, PTRH1, FCGR2C, PDK1, ZAP70, NUDT14, PASK, PHKB, IRF8, PAFAH2, GFER, ATP6V1E2, RP11-5106.1, CNPY4, OR2A9P, H3F3AP4, EPHX2, ALDH16A1, RPLP1, KDELC1, QPCTL, NCALD, CD27, TBCID9, CD96, RPS6KB1, IKBKG, GPA33, ETF1, AC061992.2, NR2E1, TEX264, FNDC3A, LRRC16A, SLC39A8, FXN, YDJC, LRRN3, ANXA6, SATB2, PDE3B, MIR22HG, NELFCD, KLHL5, GRIPAP1, FLYWCH2, ATP6V1F, CTBP1, PPIAL4C, ZNF223, ZSCAN21, AC009237.8, CFAP97, NCAM1, FAM53B, SLC25A6, TTLL7, RPL7A, ACADS, MAPK11P1L, PLAU, EPB41L4B, WRAP53, SNTB1, EIF3G, NECAB1, ZFP92, EPC2, DOCK10, SULF2, MIR29A, AHNAK, SPTBN1, PELO, HLA-DRA, RAPGEF4, ALKBH6, MRPL37, MRPS11, JMJD7, CXCR2, ZNF595, TWSG1, FAM58A, EIF4B, GOSR1, ATP5A1, FZD5, ZNF580, CST3, RPS4X, FLNB, C9orf142, INPP4A, ITM2B, TTC7A, DDX24, GNLY, HIP1R, GDF15, ZDHHC19, BIN2, LAX1, SAMD12, ACSS1, FFAR3, NSMCE2, ID2, LYZ, TLE1, C16orf74, PRTN3, OXNAD1, RPS6KA5, MRPL55, PNMA3, INO80E, ABHD17A, SLC28A3, OSM, SLC05A1, ATP5G2, WEE2-AS1, KLRDI, PARPI, CCDC62, APOBEC3H, R3HCC1, CHMP7, EIFI, RFTNI, CHACI, RPI1-443P15.2, TBX21, ERFE, PLOD2, ZNF543, HMGBIP5, ITK, TBCIDI0C, AP000476.1, ADA, PHF20L1, ASGR2, DCPS, AKRIBI, SPN, PLPP3, CHD6, PRMT6, C3AR1, TBCID8B, CDK5RAP3, MIR3142HG, CDS, EVAIC, CD74, PLAC8, GPAT2, LINC00426, HINT2, PAOX, TUTI, GPBARI, BINI, ZCCHC18, GRAMDIC, AF27936.9, CD4, XPC, EEFIG, ACSF2, SCD, ZNF772, CCDC102A, NRIH2, METTL13, ARHGEFI0L, ADCK2, USP28, FAM66C, SIGLEC5, KANSLIL, SERPINEI, RLNI, CNOT6, and IKZF3.

33. (canceled)

34. The method of claim 32, wherein the subject is suffering from a viral infection, a non-viral infection, or inflammation or traumatic injury.

35-39. (canceled)

40. The method of any one of claims 1, 2, 8, 13, 25, 26, and 28, wherein the gene is ADAM9 gene.


SEQUENCE LISTING (4 sequences)

SEQ ID NO: 1 - DNA, 25 bp, Artificial Sequence
  Synthetic Primer
ggactcagaggattgctgcatttag

SEQ ID NO: 2 - DNA, 25 bp, Artificial Sequence
  Synthetic Primer
cttcgaagtagctgagtcatgctgg

SEQ ID NO: 3 - DNA, 22 bp, Artificial Sequence
  Synthetic Primer
ggtgaaggtcggagtcaacgga

SEQ ID NO: 4 - DNA, 22 bp, Artificial Sequence
  Synthetic Primer
gagggatctcgctcctggaaga